(19) United States
(12) Patent Application Publication
(10) Pub. No.: US 2024/0401044 A1
(43) Pub. Date: Dec. 5, 2024

(54) EXPLOITING IL33 SECRETION AS A THERAPEUTIC TARGET IN CANCER

(71) Applicant: ROSWELL PARK CANCER INSTITUTE CORPORATION HEALTH RESEARCH, INC., Buffalo, NY (US)

(72) Inventors: Prasenjit DEY, Clarence, NY (US); Aftab ALAM, Clarence, NY (US)

(21) Appl. No.: 18/688,261

(22) PCT Filed: Aug. 30, 2022
(86) PCT No.: PCT/US2022/075660

§ 371 Date: Feb. 29, 2024

Related U.S. Application Data
(60) Provisional application No. 63/238,531, filed on Aug. 30, 2021.

(51) Int. Cl. C12N 15/113; A61K 31/7048; A61K 45/06; A61P 35/00; C12N 9/22; C12N 15/11

(52) U.S. Cl. CPC C12N 15/113; A61K 31/7048; A61K 45/06; A61P 35/00; C12N 9/22; C12N 15/111; C12N 2310/122; C12N 2310/20

(57) ABSTRACT

Disclosed herein is the effect that IL-33 has on the tumor microenvironment. In one aspect, disclosed herein are methods for treating a cancer, inhibiting T_{H}2 pro-tumorigenic cytokines in the TME, inhibiting secretion of IL-33 in the TME, and/or inhibiting type 2 immune cell infiltration in the TME by administering of an antifungal agent, a MEK inhibitor, an IL-33 inhibitor, or an ST2 inhibitor, or any combination thereof.

This application claims the benefit of U.S. Provisional Application No. 63/238,531, filed on Aug. 30, 2021 which is incorporated herein by reference in its entirety.

This invention was made with government support under Grant No. CA218891 and CA262822 awarded by the National Cancer Institute. The government has certain rights in the invention.

### I. BACKGROUND

Pancreatic ductal adenocarcinoma (PDAC) is associated with a distinctive tumor immune profile that consists mostly of immunosuppressive immune cells, such as tumor-associated macrophages (TAMs), T regulatory (Treg) cells, CD4+ TH2 cells and myeloid-derived suppressor cells (MDSCs) which act in concert to inhibit effector T cell activation, expansion and function, thereby contributing to PDAC progression. TH2 cells infiltrate the pancreas in the early stages of tumorigenesis and secrete type 2 cytokines [interleukin (IL4) and IL13)] that promote metabolic reprogramming and proliferation of cancer cells in murine Kras*-driven PDAC. Consistent with type 2 immune responses driving PDAC progression in mouse models, PDAC patients with predominantly TH2 (CD45+CD3+CD4+Gata3+)-polarized lymphoid cell tumor infiltration exhibit reduced survival, compared to patients with a higher tumor infiltration of TH1 (CD45+CD3+CD4+Tbet+) cells. Moreover, the circulating levels of IL4 negatively correlate with disease-free survival in PDAC patients. Despite the significant impact type 2 immune cells have on the tumor microenvironment (TME), the mechanisms by which type 2 immune cells traffic to the TME are unknown. What are needed are new therapeutic agents that can prevent the activation and infiltration of type 2 immune cells into the TME.

### II. SUMMARY

Disclosed are methods and compositions related to inhibition of IL-33 production and/or signaling through the IL-33 receptor in the treatment of cancer.

In one aspect, disclosed herein are methods of treating, inhibiting, reducing, decreasing, ameliorating and/or preventing a cancer and/or metastasis (such as, for example, pancreatic cancer, colon cancer, and lung cancer) comprising a KRAS^{G12D} substitution in a subject comprising administering to the subject an antifungal agent (including, but not limited to an antifungal agent that inhibits an Alternaria alternata and/or Malassezia globosa infection in the tumor microenvironment, such as, for example, natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and/or voriconazole), a MEK inhibitor (such as, for example, CI-1040, PD0325901, binimetinib, cobimetinib, selumetinib, and/or Trametinib), an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof. In one aspect, the antifungal agent is administered prior to the onset of pancreatitis.

Also disclosed herein are methods of treating, inhibiting, reducing, decreasing, ameliorating and/or preventing a cancer and/or metastasis of any preceding aspect, wherein the method further comprises administering to the subject an anti-cancer agent.

In one aspect, disclosed herein are methods of inhibiting TH2 pro-tumorigenic cytokines (such as, for example, IL4, IL5, and/or IL13) in a tumor microenvironment of a cancer (such as, for example, pancreatic cancer, colon cancer, and lung cancer) in a subject, the method comprising administering to the subject an antifungal agent (including, but not limited to an antifungal agent that inhibits an Alternaria alternata and/or Malassezia globosa infection in the tumor microenvironment, such as, for example, natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and/or voriconazole), a MEK inhibitor (such as, for example, CI-1040, PD0325901, binimetinib, cobimetinib, selumetinib, and/or Trametinib), an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof. In one aspect, the antifungal agent is administered prior to the onset of pancreatitis.

Also disclosed herein are methods of decreasing secretion of IL-33 in a tumor microenvironment of a cancer (such as, for example, pancreatic cancer, colon cancer, and lung cancer) in a subject, the method comprising administering to the subject an antifungal agent (including, but not limited to an antifungal agent that inhibits an Alternaria alternata and/or Malassezia globosa infection in the tumor microenvironment, such as, for example, natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and/or voriconazole), a MEK inhibitor (such as, for example, CI-1040, PD0325901, binimetinib, cobimetinib, selumetinib, and/or Trametinib), an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof. In one aspect, the antifungal agent is administered prior to the onset of pancreatitis.

In one aspect, also disclosed herein are methods of decreasing infiltration of Type 2 immune cells (such as, for example, innate lymphoid cells (ILC) 2 (ILC2) or T_{H}2 cells) in a tumor microenvironment of a cancer (such as, for example, pancreatic cancer, colon cancer, and lung cancer) in a subject, the method comprising administering to the subject an antifungal agent (including, but not limited to an antifungal agent that inhibits an Alternaria alternata and/or Malassezia globosa infection in the tumor microenvironment, such as, for example, natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and/or voriconazole), a MEK inhibitor (such as, for example, CI-1040, PD0325901, binimetinib, cobimetinib, selumetinib, and/or Trametinib), an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof. In one aspect, the antifungal agent is administered prior to the onset of pancreatitis.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, 1G, 1H, 1I, 1J, 1K, 1L, 1M, and 1N show that type 2 immune cell infiltration increases significantly in PDAC tumor microenvironment. FIG. 1A shows a Schematic diagram showing strategy for KPC (Kras^{G12D};p53^{R172H};pdx-Cre) PDAC mouse modeling. FIG. 1B shows flow cytometry gating strategy and frequency of T_{H}2 cells out of total CD4 T cells in normal pancreas, spleen and PDAC tumor. FIG. 1C shows representative flow cytometry histogram of TH2 cell phenotype stained with either isotype control (blue histogram) and CD4, Gata3 and CCR4 antibodies (red histogram). FIG. 1D shows the frequency of T_{H}2 cells out of total CD4 T cells in normal pancreas, spleen and PDAC tumor (n=3). FIG. 1E shows the gating strategy and frequency of ILC2 cells out of total Lineage negative (CD3, Ly6G, Ly6C, CD11b, CD45R/B220 and TER-119) cells in normal pancreas, bone marrow, spleen and PDAC tumor (n=3). FIG. 1F shows representative flow cytometry histogram of sorted ILC2s stained with either either isotype control (blue histogram) and ST2, Sca-1 and CD127 antibodies (red histogram). FIG. 1G shows the of ILC2 cells out of total Lin cells in normal pancreas, bone marrow, spleen and PDAC tumor, (n=4). FIG. 1H shows the frequency of ILC2 cells out of total Lin-cells in normal pancreas, PanIN and PDAC tumor, (n=4). FIG. 1I shows a schematic showing experimental strategy for single-cell RNA sequencing (scRNA-seq) from PDAC tumor-bearing mice. CD45⁺ cells were flow-sorted from PDAC tumor and 10,000 live CD45⁺ cells were used for scRNA-seq. FIG. 1J shows t-SNE plot of immune cells showing 14 clusters belonging to 3 major groups in PDAC sample. FIG. 1K Bar graph showing the proportion of major immune cell clusters in PDAC sample. FIG. 1L shows t-SNE plots showing T_{H}2 lineage genes (Cd4, Gata3 and Ccr4) expression in sub-cluster of immune cells. The color key bar represents gene expression level. FIG. 1M shows t-SNE plots showing expression of ILC2 lineage genes (Hes1, Hs3st1 and Il1rl1) expression in sub-cluster of immune cells. The color key represents gene expression level. FIG. 1N shows gating and frequency of ILC2 out of total lineage negative (CD3, CD14, CD16, CD19, CD20, and CD56) cells in human PDAC tumor. Data are combined from three independent experiments and are presented as mean ±SEM. P values were calculated using Student t-test. ns, no significance.

FIGS. 2A, 2B, 2C, 2D, 2E, 2F, and 2G show type2 cell infiltration in PDAC tumors. FIG. 2A shows the gating strategy of TH2, ILC2 and eosinophil. FIG. 2B shows the frequency of TH2 cells out of total CD45 cells in normal pancreas, spleen, and PDAC tumor (n=3). FIG. 2C shows the frequency of ILC2 out of total CD45 cells in spleen, bone marrow, small intestine, and pancreas, (n=3) in KPC mice compared to control C57 BL/6 mice. FIG. 2D shows the frequency of ILC2 out of total CD45 cells in normal pancreas (n=3) and PDAC (n=4) in iKPC mice compared to control C57BL/6 mice. FIG. 2E shows the frequency of eosinophil out of total CD45 cells in spleen, bone marrow, blood, normal pancreas, and PDAC tumor (n=3). FIGS. 2F and 2G shows t-SNE plots showing frequency of TH2 and ILC2 clusters from scRNA seq data set. Results are shown as mean ±SEM. P values were calculated using Student t test.

FIGS. 3A, 3B, 3C, 3D, 3E, 3F, 3G, 3H, 3I, 3J and 3K show that IL33 is a downstream target of oncogenic Kras*: FIG. 3A is a schematic showing doxycycline inducible KRAS^{G12D}(iKras*) transgenic mouse model (iKPC). Strategy to turn ON and OFF Kras signaling in cell lines followed by transcriptome analysis. FIG. 3B shows GSEA analysis of RNAseq dataset comparing Kras ON vs Kras OFF. FIG. 3C shows GSEA analysis of RNAseq dataset showing enrichment of hallmark Kras signaling comparing Kras ON vs Kras OFF. FIG. 3D shows a heatmap of gene list comparing Kras ON, OFF-2 and 4 days in 4 murine cell lines. Red arrow showing IL33 gene. FIG. 3E shows RT-qPCR analysis of IL33 expression in the Kras ON, OFF-2 and 4 days. FIG. 3F shows Western blot analysis of IL33 and P-p42/44 in Kras ON, OFF-1, 2 and 3 days in murine cell line. FIG. 3G shows Western blot analysis of IL33 and P-p42/44 in Kras ON, OFF and re-ON in murine cell line. FIG. 3H shows Western blot analysis of IL33, P-p42/44 (P-ERK1/2) and P-Akt upon treatment with MEK inhibitors (CI-1040 and Trametinib) in murine cell line. β-actin acts as a loading control. FIG. 3I shows representative confocal images of IL33 and αSMA staining in mouse PDAC tumor which displays exclusive expression of IL33 in the cancer cells. Magnification 63×. FIG. 3J shows representative IHC images of IL33 in human PDAC tumor. Inset (red box) showing 100× magnification. (n=121). FIG. 3K shows statistical analysis of IL33 staining of human PDAC TMA. The expression of the protein within tumor cells or normal epithelial cells were evaluated. The intensity of IHC staining was scored as negative (0), weak (1), medium (2), and strong (3). Scale bar 100 μm, unless indicated otherwise. Data are combined from three independent experiments and are presented as mean ±SEM., P values were calculated using Student t-test. ns, no significance. GSEA: Gene signature enrichment analysis.

FIGS. 4A, 4B, 4C, 4D, 4E, and 4F show that Kras drives IL33 expression in PDAC. FIG. 4A shows a schematic showing strategy for doxycycline inducible iKPC cell line AK-B6 and AK4298 for quantitative realtime analysis. FIG. 4B shows quantitative real-time analysis of AK-B6 and AK4298 cell line after Kras ON and OFF conditions (n=3). FIG. 4C shows a Western blot showing expression of IL33, pERK1/2, pAKt-S307 upon treatment with MEK (CI1040 and trametinib) and AKT (Buparlisib and GSK-690696) inhibitors for 24 hrs. β-actin was used as loading control. FIG. 4D shows representative tables for human PDAC TMA IHC analysis showing IL33 expression profile and statistical analysis of adjacent normal (n=63) vs PDAC tumor (n=93). FIG. 4E shows representative plots showing oncomine data set analysis for IL33 expression in normal vs PDAC tumors. FIG. 4F shows a representative plot showing IL33 expression profile in different human tissues (Genotype tissue expression [GTEx]). Results are shown as mean ±SEM. P values were calculated using Student t test. Chi-square test and spearman's correlation analysis was done for human PDAC TMA for statistical significance.

FIGS. 5A, 5B, 5C, 5D, 5E, 5F, 5G, 5H, 5I, 5J and 5K show that IL33 is required for recruitment of type 2 immunocytes: FIG. 5A shows IL33 gene expression was determined by RT-qPCR relative to β-actin in non-target (shCtrl) vs shIL33 (#1 and #2) stable murine cell line. FIG. 5B shows Western blot analysis showing knockdown of IL33 in shIL33 (#1 and #2) stable murine cell line. β-actin was used as loading control. FIG. 5C shows intrapancreatic injection was done to transplant shCtrl (n=23) and shIL33 (#1 [n=25] and #2 [n=24]) stable PDAC isogenic mouse cell line. Representative bioluminescence images showing orthotopic transplanted PDAC tumors. FIG. 5D shows Kaplan-Meier survival curves of mice orthotopically transplanted with shCtrl and shIL33 (#1 and #2) stable PDAC isogenic mouse cell line (n=10). FIG. 5E shows representative confocal images showing epithelial cell-specific nuclear expression of IL33 (green) in orthotopic transplanted control and shIL33 PDAC tumors. PanCK (red) was used as epithelial cell marker, DAPI (blue) was used to stain nucleus. Magnification 63×. FIG. 5F shows a schematic showing a accumulation of ascites fluid on day 25-28 of orthotopically transplanted shCtrl and shIL33 PDAC tumor-bearing mice (left). IL-33 was quantified in ascites fluid using ELISA (n=3) (right). FIG. 5G shows the frequency of ILC2s in orthotopically transplanted shCtrl and shIL33 PDAC tumors relative to total Lin cells. FIG. 5H shows the frequency of T_{H}2 in orthotopically transplanted shCtrl and shIL33 PDAC tumors relative to total CD4⁺ cells. FIG. 5I shows the frequency of T_{regs} in orthotopically transplanted shCtrl and shIL33 PDAC tumors relative to total CD4⁺ cells. FIG. 5J shows a schematic showing flow sorting of ILC2 cells from orthotopically transplanted shCtrl and shIL33 PDAC tumors (left). qRT-PCR analysis was performed for ILC2 lineage signature genes Tph1, Ill3, Il5, and Areg (right). Data are combined from three independent experiments and are presented as mean ±SEM., P values were calculated using Student t-test. ns, no significance.

FIGS. 6A, 6B, 6C, 6D, 6E, 6F, 6G, 6H, and 6I show IL33 depletion delays tumor progression. FIG. 6A shows colony formation assay of shCtrl and shIL33 stable iKPC cell lines. Cells were fixed and stained with crystal violet and colonies were quantified using image J (n=4). FIG. 6B shows a representative image of orthotopic transplanted shCtrl and shIL33 KPC pancreatic tumors (n=5). FIG. 6C is a bar graph showing tumor weight of shCtrl and shIL33 orthotopically transplanted KPC pancreatic tumors (n=5). FIG. 6D shows the frequency of TH2 in orthotopic transplanted shCtrl and shIL33 in KPC pancreatic tumors relative to total CD45 positive cells. FIG. 6E shows the frequency of ILC2s in orthotopic transplanted shCtrl and shIL33 in KPC pancreatic tumors relative to total CD45 positive cells. FIG. 6F is a western blot showing IL33 expression in CRISPR/Cas9 control and CRISPR/Cas9 IL33 knockout cells. β-actin was used as loading control. FIG. 6G shows a representative image of orthotopic transplanted CRISPR/Cas9 control and IL33 knockout pancreatic tumors (n=5). FIG. 6H is a bar graph showing tumor weight of CRISPR/Cas9 control vs IL33 knockout orthotopically transplanted PDAC tumors (n=5). FIG. 6I shows the frequency of ILC2, TH2, Treg, CD8, neutrophils, MDSC and B cells out of total CD45 cells in control and IL33 knockout orthotopically transplanted PDAC tumors and peripheral blood (n=3-5). Results are shown as mean ±SEM. P values were calculated using Student t test.

FIGS. 7A, 7B, 7C, 7D, 7E, 7F, 7G, 7H, 7I, and 7J show intratumor fungi facilitate the release of IL33 from PDAC cells: FIG. 7A shows representative IHC images of IL33 in normal spleen, normal pancreas, PanIN from KC mice and KPC PDAC 6, 12- and 24-weeks old mice. FIG. 7B shows fluorescence images showing nuclear expression of IL33 (green, white arrows) in PDAC cell line. DAPI (blue) was used for nuclear staining, Magnification 40×, Scale 75 μm. FIG. 7C shows subcellular fractionation of inducible murine PDAC cell line exhibiting IL33 expression in cytoplasm and nucleus. Lamin A/C and β-tubilin were used as nuclear and cytoplasmic loading control respectively. FIG. 7D shows 18S rRNA sequence of fungal species in normal pancreas and PDAC tumors. The heatmap of relative abundancies of fungal genus and family in gut and PDAC. FIG. 7E shows fluorescence in-situ hybridization (FISH) showing fungal population in normal pancreas and PDAC. D223 fungal specific probe was used to detect the fungal species in normal pancreas. FIG. 7F shows schematic showing strategy of fungal extract treatment followed by biochemical assay to determine IL33 expression in cells treated with Alternaria conditioned media. FIG. 7G shows western blot analysis of IL33 in control PDAC cell line treated with fungal extract (Alternaria) for different time points (2 h, 3 h, 6 h, and 24 h) and shIL33 PDAC cell line. β-actin was used a loading control. FIG. 7H shows that IL33 was measured in conditioned media using ELISA in PDAC cell line treated with Alternaria extract for different time points (2 h, 3 h and 6 h). FIG. 7I shows a schematic showing strategy for quantification of IL5 in flow sorted ILC2 cultured with PDAC cell conditioned media treated with fungal extract. (FIG. 7J shows IL5 from flow sorted ILC2s was measured using ELISA. Scale bar 100 μm, unless indicated otherwise. Data are combined from three independent experiments and are presented as mean ±SEM., P values were calculated using Student t-test. ns, no significance.

FIGS. 8A, 8B, 8C, 8D, 8E, 8F, 8G, 8H, 8I, 8J, and 8K show fungal mediated release of IL33 from PDAC cell lines. FIG. 8A is an illustration showing the release of IL33 and its unknown mechanism. FIG. 8B shows 18S rRNA sequence of fungal species in normal pancreas and PDAC tumors. The heatmap of relative abundancies of fungal species in gut and PDAC. FIGS. 8C and 8E are western blots showing IL33 expression in a time course experiment with Alternaria alternata extract treatment in PDAC cell lines PJ/B6-4298 and AK192. FIGS. 8D and 8F show mouse IL33 ELISAs for quantification of IL33 in spent media of PDAC cell line PJ/B6-4298 and AK192 upon Alternaria alternata extract treatment. FIG. 8G shows a western blot showing IL33 expression in a time course experiment with Aspergillus extract treatment in AK-B6 PDAC cell line. FIG. 8H shows mouse IL33 ELISA for quantification of IL33 in spent media of AKB6 PDAC cell line upon Aspergillus extract treatment. FIG. 8I is a western blot showing IL33 expression in a time course experiment with Candida extract treatment in AKB6 PDAC cell line. FIG. 8J shows a mouse IL33 ELISA for quantification of IL33 in spent media of AK-B6 PDAC cell line upon Candida extract treatment. FIG. 8K shows confocal images showing IL33 release from AKB6 PDAC cell line upon Alternaria alternata extract treatment. β-actin was used as loading control for all the western blots. Results are shown as mean ±SEM. P values were calculated using Student t test.

FIGS. 9A, 9B, 9C, 9D, 9E, 9F, 9G, 9H, 9I, 9J, and 9K show intratumor fungus accelerates PDAC tumor growth: FIG. 9A shows a schematic showing fungal depletion strategy, mice were treated with 5 consecutive doses of amphotericin (200 ug/dose). Followed by 3 weeks of amphotericin (0.5ug/ml) treatment in drinking water. After 3 weeks, PDAC cells were orthotopically transplanted and tumor progression studies were performed after 28-35 days. FIG. 9B shows representative MRI images showing orthotopically transplanted shCtrl and shIL33 PDAC tumors with their relative volumes (n=5). Red circles define the tumor boundaries. FIG. 9C shows a bar graph showing tumor volume of orthotopically transplanted shCtrl and shIL33 PDAC tumors (n=14) and shIL33 (#1 [n=9] and #2 [n=10]) with or without amphotericin B treatment. FIG. 9D shows Kaplan-Meier survival curves of mice orthotopically transplanted with shCtrl and shIL33 PDAC tumors with or without antifungal treatment (n=10). FIG. 9E shows the frequency of ILC2 in orthotopic transplanted shCtrl and shIL33 PDAC tumors treated with or without antifungal relative to total CD45 positive cells. FIG. 9F shows the frequency of T_{H}2 cells in orthotopic transplanted shCtrl and shIL33 PDAC tumors treated with or without antifungal relative to total CD45 positive cells. FIG. 9G shows schematic showing fungal transplantation strategy, mice were treated with 5 consecutive doses of amphotericin (200 ug/dose). Followed by 3 weeks of amphotericin (0.5ug/ml) treatment in drinking water. After 3 weeks fungus (Alternaria alternata and Malassazia globose, 10⁸ CFU/ml) was transplanted in mice. After 7 days of fungus transplantation, PDAC cells were orthotopically transplanted and tumor progression studies were performed after 28-35 days. FIG. 9H shows representative IHC and immunofluorescence images showing IL33 expression in control and fungal transplanted PDAC tumors (n=10), Magnification 40×. FISH showing fungal colonization in fungal transplanted PDAC tumors FIG. 9I shows 18S rRNA sequencing showing fungal species in PDAC tumors, Alternaria Alternata and Malassezia globosa rechallenge experiments. Also, shown are the 18S rRNA sequencing in stool samples. Positive control-Alternaria is a sample of pure Alternaria culture. FIG. 9J shows is a bar graph showing the wet weight of the control, antifungal and fungal transplanted PDAC tumors (n=10). FIG. 9K shows the frequency of ILC2s in fungal transplanted orthotopic PDAC tumors treated with or without antifungal relative to total CD45 positive cells. Scale bar 100 μm, unless indicated otherwise. Data are combined from three independent experiments and are presented as mean ±SEM, P values were calculated using Student t-test. ns, no significance.

FIGS. 10A, 10B, 10C, 10D, 10E, 10F, 10G, 10H, 10I, and 10J show that the mycobiome promote PDAC tumor progression: FIG. 10A shows representative bioluminescence images showing orthotopic transplanted with shCtrl and shIL33 (#1 and #2) stable PDAC isogenic mouse cell line with or without antifungal treatment (n=5). FIG. 10B shows western blot showing IL33 expression in orthotopically transplanted tumor lysate from shCtrl and shIL33 (#1 and #2) stable PDAC isogenic mouse cell line and amphotericin B treatment. FIG. 10C shows representative image showing orthotopically transplanted isogenic mouse cell line tumors with or without fungal repopulation (n=5). FIGS. 10D, 10E, and 10F show 18S sequencing data showing gut and intratumor fungal abundancies as species, families and class. FIGS. 10G, 10H, 10I, and 10J show the frequency of TH2 out of total CD4 cells, frequency of CD4, B and CD8 cells in Alternaria alternata and Malassezia globose rechallenge experiments after fungal depletion in orthotopically transplanted isogenic AKB6 PDAC tumors in C57BL/6 mice (n=5). Results are shown as mean ±SEM. P values were calculated using Student t test.

FIGS. 11A, 11B, 11C, 11D, 11E, 11F, 11G, 11H, 11I, and 11J show that IL33 mediated ILC2 recruitment is necessary for tumor progression.: FIG. 11A shows a schematic showing strategy for orthotopic co-transplantation of PDAC and ILC2 cells. FIG. 11B shows representative MRI scans showing axial images of CRISPR-Cas9 knockout tumors (IL33 WT vs IL33 KO), with or without ILC2 co-transplantation. Fgirue 11C shows a bar graph showing tumor volume calculated by MRI image analysis (n=5-7). FIG. 11D is a picture showing gross PDAC tumor with or without ILC2 in IL33 WT vs IL33 KO mice (n=5). FIG. 11E is a bar graph showing PDAC tumor wet weight with or without ILC2 in IL33 WT vs IL33 KO mice (n=5-7). FIG. 11F is a schematic showing fungal activation pathway, where dectin-1 receptor ligates fungal components and induce Src-Syk-CARD9 signaling cascade. FIG. 11G is a histogram showing dectin-1 expression on PDAC cell line, analyzed by flow cytometry (n=3). FIG. 11H is a western blot showing expression of pSrc, Src, pSyk, Syk, CARD9, p-NFκB and IL-33 upon treatment with Alternaria alternata. β-actin was used as a loading control. FIG. 11I shows a representative IHC image showing CARD9 expression in orthotopic transplanted tumor with or without antifungal treatment. Scale bar 100 μm. FIG. 11J is a working model showing fungus mediated secretion of IL-33 from PDAC tumor, attracting type 2 immune cells (ILC2, TH2, and Treg) thereby promoting tumor progression. Results are shown as mean ±SEM. P values were calculated using Student t-test.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder, preventative treatment, that is, treatment directed to minimizing or partially or completely inhibiting the development of the associated disease, pathological condition, or disorder, and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Using the Compositions

Pancreatic ductal adenocarcinoma (PDAC) is associated with a distinctive tumor immune profile that consists mostly of immunosuppressive immune cells, such as tumor-associated macrophages (TAMs), T regulatory (T_{reg}) cells, CD4⁺ T_{H}2 cells and myeloid-derived suppressor cells (MDSCs) which act in concert to inhibit effector T cell activation, expansion and function, thereby contributing to PDAC progression. We show herein a unique metabolic mechanism by which CD4⁺ T_{H}2 cells support PDAC progression. We found that T_{H}2 cells infiltrate the pancreas in the early stages of tumorigenesis and secrete type 2 cytokines [interleukin (IL4) and IL13)] that promote metabolic reprogramming and proliferation of cancer cells in murine Kras*-driven PDAC. Consistent with type 2 immune responses driving PDAC progression in mouse models, PDAC patients with predominantly TH2 (CD45⁺CD3⁺CD4⁺Gata3⁺)-polarized lymphoid cell tumor infiltration exhibit reduced survival, compared to patients with a higher tumor infiltration of T_{H}1 (CD45⁺CD3⁺CD4⁺Tbet⁺) cells. Moreover, the circulating levels of IL4 negatively correlate with disease-free survival in PDAC patients.

While the T_{H}2 cell-secreted cytokines, IL4 and IL13, promote anabolic growth of PDAC cancer cells, we sought to determine the molecular mechanisms driving the recruitment and expansion of these T_{H}2 cells in the tumor microenvironment (TME). Herein we identified oncogenic KRAS-mediated upregulation of IL33, which is a known potent activator of T_{H}2, innate lymphoid cells 2 (ILC2), eosinophils, T_{regs}, and basophils. IL33 is both a damage-associated molecular pattern (DAMP) and a cytokine that belongs to the IL1 cytokine superfamily, which plays important roles in innate immunity, inflammation and tumor development. IL33 exerts its biological function by binding to its cognate receptor, suppression of tumorigenicity (ST)2 (also called IL1RL1), which interacts with its co-receptor, the IL1 receptor accessory protein (IL1RAcP). Both receptors are expressed by innate and type 2 immune cells that include TH2 cells, ILC2s, eosinophils, T_{reg}, and mast cells. Accordingly, in one aspect, disclosed herein are methods of inhibiting T_{H}2 pro-tumorigenic cytokines (such as, for example, IL4, IL5, and/or IL13) in a tumor microenvironment of a cancer (such as, for example, pancreatic cancer, colon cancer, and lung cancer) in a subject, the method comprising administering to the subject an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof.

ILC2s are the most prominent target of IL33 and stimulate activation of ILC2 in response to a number of stimuli, such as allergens and parasites. ILC2s are primarily tissue-resident immunocytes that remain in close proximity to the epithelial cells, enabling ILC2 cells to respond to an immune insult within hours by producing cytokines such as IL4, IL13 and IL5 that in turn activate additional players of the type 2 immune response. Moreover, activation of ILC2 cells is independent of antigen presentation and uses ligand receptors often specific to the tissue where they reside. Thus, disclosed herein are methods of decreasing infiltration and/or activation of Type 2 immune cells (such as, for example, innate lymphoid cells (ILC) 2 (ILC2) or T_{H}2 cells) in a tumor microenvironment of a cancer (such as, for example, pancreatic cancer, colon cancer, and lung cancer) in a subject, the method comprising administering to the subject an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof.

Because of the negative effect IL-33 can have on the tumor microenvironment in terms of infiltration and/or activation of pro-tumorigenic Type 2 immune cells and secretion of pro-tumorigenic cytokines (e.g., IL-4, IL-5, and/or IL-13) by said cells, it is advantageous to a subject with a cancer to suppress IL-33 in the TME. Therefore, disclosed herein are methods of decreasing, inhibiting, reducing, ameliorating, and/or preventing secretion of IL-33 in a tumor microenvironment of a cancer (such as, for example, pancreatic cancer, colon cancer, and lung cancer) in a subject, the method comprising administering to the subject an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof.

Gut microbes can interact with the host and modulate disease pathogenesis and response to therapy. Microbes can colonize in the pancreas and play a role in PDAC tumorigenesis and progression. Specifically, the fungal-biome (mycobiome) present in the gut lumen migrates to the pancreas via the sphincter of Oddi. The translocation of endoluminal fungi to the pancreas allows the fungal population to increase by >3000-fold in PDAC compared to the normal pancreas.

Herein is shown that oncogenic Kras was shown to induce IL33 expression and secretion by cancer cells through a pathway that depends on fungi within the TME and that genetic deletion of IL33 or anti-fungal treatment each leads to robust PDAC tumor regression. This novel mechanism of cooperative interactions of resident fungi with Kras-IL33 and priming type 2 immune responses to accelerate tumor progression identifies novel therapeutic strategies for PDAC. Accordingly, inhibition of fungi in the TME (i.e., administration of an antifungal) can also be used in the methods of inhibiting T_{H}2 pro-tumorigenic cytokines in the TME of a cancer, methods of decreasing secretion of IL-33 in a tumor microenvironment of a cancer, and methods of decreasing infiltration and/or activation of Type 2 immune cells (such as, for example, innate lymphoid cells (ILC) 2 (ILC2) or T_{H}2 cells) in a tumor microenvironment of a cancer disclosed herein. Accordingly disclosed herein are methods of inhibiting, decreasing, reducing, and/or preventing T_{H}2 pro-tumorigenic cytokines (such as, for example, IL4, IL5, and/or IL13) in a tumor microenvironment of a cancer (such as, for example, pancreatic cancer, colon cancer, and lung cancer) in a subject, the method comprising administering to the subject an antifungal agent. Also disclosed herein are methods of decreasing, inhibiting, reducing, and/or preventing secretion of IL-33 in a tumor microenvironment of a cancer (such as, for example, pancreatic cancer, colon cancer, and lung cancer) in a subject, the method comprising administering to the subject an antifungal agent. Further, disclosed herein are methods of decreasing, inhibiting, reducing, ameliorating, and/or preventing infiltration and/or activation of Type 2 immune cells (such as, for example, innate lymphoid cells (ILC) 2 (ILC2) or T_{H}2 cells) in a tumor microenvironment of a cancer (such as, for example, pancreatic cancer, colon cancer, and lung cancer) in a subject, the method comprising administering to the subject an antifungal agent.

As shown herein, not all fungal infiltration in the TME leads to IL-33 secretion. Candida infection did not increase IL-33 production and, thus, specific inhibition of Candida did not have an effect on the cytokines or cell types in the TME. However, Alternaria spp (such, as, for example, Alternaria alternata) and Malassezia spp (such, as, for example, Malassezia globosa) do play a role in IL-33 secretion and inhibition of Alternaria spp and/or Malassezia spp. had a therapeutic effect. Accordingly, the antifungal agent for use in the disclosed methods can be any antifungal agent that is effective in inhibiting Alternaria spp (such, as, for example, Alternaria altemata) and Malassezia spp (such, as, for example, Malassezia globosa) infection in the TME. The antifungal agent can be a broad spectrum antifungal agent or one specifically tailored to the inhibition of Alternaria spp (such, as, for example, Alternaria alternata) and Malassezia spp (such, as, for example, Malassezia globosa). Examples of antifungals that can be used in the disclosed methods include, but are not limited to natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and/or voriconazole. In some cases antifungals are administered to cancer patients to treat pancreatitis caused by a fungal infection. However, by administering an antifungal before any symptoms of pancreatitis are present, not only is the pancreatitis prevented, but surprising benefit of inhibiting IL-33 secretion in the TME is realized. Thus, in one aspect, the antifungal agent is administered prior to the onset and/or diagnosis of pancreatitis.

As shown herein, treatment with inhibitors (CI-1040, Trametinib) of MEK, a downstream target of Kras signaling, resulted in complete inhibition of IL33 expression in these cell lines. Thus, it is understood and herein contemplated that administration of MEK inhibitors (including, but not limited to CI-1040, PD0325901, binimetinib, cobimetinib, selumetinib, and/or Trametinib) alone or in combination with an antifungal agent, IL-33 inhibitor, and or ST2 inhibitor can be used in the methods of inhibiting T_{H}2 pro-tumorigenic cytokines in the TME of a cancer, methods of decreasing secretion of IL-33 in a tumor microenvironment of a cancer, and methods of decreasing infiltration and/or activation of Type 2 immune cells (such as, for example, innate lymphoid cells (ILC) 2 (ILC2) or T_{H}2 cells) in a tumor microenvironment of a cancer disclosed herein. In one aspect, disclosed herein are methods of treating, inhibiting, reducing, decreasing, ameliorating and/or preventing T_{H}2 pro-tumorigenic cytokines (such as, for example, IL4, IL5, and/or IL13) in a tumor microenvironment of a cancer (such as, for example, pancreatic cancer, colon cancer, and lung cancer) in a subject, the method comprising administering to the subject an antifungal agent (including, but not limited to an antifungal agent that inhibits an Alternaria alternata and/or Malassezia globosa infection in the tumor microenvironment, such as, for example, natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and/or voriconazole), a MEK inhibitor (such as, for example, CI-1040, PD0325901, binimetinib, cobimetinib, selumetinib, and/or Trametinib), an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof. Also disclosed herein are methods of treating, inhibiting, reducing, decreasing, ameliorating and/or preventing secretion of IL-33 in a tumor microenvironment of a cancer (such as, for example, pancreatic cancer, colon cancer, and lung cancer) in a subject, the method comprising administering to the subject an antifungal agent (including, but not limited to an antifungal agent that inhibits an Alternaria alternata and/or Malassezia globosa infection in the tumor microenvironment, such as, for example, natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and/or voriconazole), a MEK inhibitor (such as, for example, CI-1040, PD0325901, binimetinib, cobimetinib, selumetinib, and/or Trametinib), an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof. Similarly, disclosed herein are methods of treating, inhibiting, reducing, decreasing, ameliorating and/or preventing infiltration and/or activation of Type 2 immune cells (such as, for example, innate lymphoid cells (ILC) 2 (ILC2) or T_{H}2 cells) in a tumor microenvironment of a cancer (such as, for example, pancreatic cancer, colon cancer, and lung cancer) in a subject, the method comprising administering to the subject an antifungal agent (including, but not limited to an antifungal agent that inhibits an Alternaria alternata and/or Malassezia globosa infection in the tumor microenvironment, such as, for example, natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and/or voriconazole), a MEK inhibitor (such as, for example, CI-1040, PD0325901, binimetinib, cobimetinib, selumetinib, and/or Trametinib), an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof.

It is understood and herein contemplated that by decreasing, inhibiting, reducing, ameliorating, and/or preventing T_{H}2 pro-tumorigenic cytokines (such as, for example, IL4, IL5, and/or IL13) in a tumor microenvironment of a cancer (such as, for example, pancreatic cancer, colon cancer, and lung cancer) in a subject; decreasing, inhibiting, reducing, and/or preventing secretion of IL-33 in a tumor microenvironment of a cancer (such as, for example, pancreatic cancer, colon cancer, and lung cancer) in a subject; and/or decreasing, inhibiting, reducing, ameliorating, and/or preventing infiltration and/or activation of Type 2 immune cells (such as, for example, innate lymphoid cells (ILC) 2 (ILC2) or T_{H}2 cells) in a tumor microenvironment of a cancer (such as, for example, pancreatic cancer, colon cancer, and lung cancer) in a subject; the end result would be the inhibition, decrease, reduction, amelioration, treatment, and/or prevention of a cancer and/or metastasis such as, for example, pancreatic cancer, colon cancer, and lung cancer) comprising a KRAS^{G12D} substitution in a subject. Thus, in one aspect, disclosed herein are methods of treating, inhibiting, reducing, decreasing, ameliorating and/or preventing a cancer and/or metastasis (such as, for example, pancreatic cancer, colon cancer, and lung cancer) comprising a KRAS^{G12D} substitution in a subject comprising administering to the subject an antifungal agent (including, but not limited to an antifungal agent that inhibits an Alternaria altemata and/or Malassezia globosa infection in the tumor microenvironment, such as, for example, natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and/or voriconazole), a MEK inhibitor (such as, for example, CI-1040, PD0325901, binimetinib, cobimetinib, selumetinib, and/or Trametinib), an IL-33 inhibitor (such as, for example, an antibody (including, but not limited to AF3625 and/or AF6326), small molecule, shRNA (including, but not limited SEQ ID NO: 13 and/04 SEQ ID NO: 14), RNAi, CRISPR/CAS9 nuclease (including, but not limited to a CRISPR/Cas9 nuclease targeting IL-33 with single guide RNA (sgRNA) comprising SEQ ID NO: 15 and/or SEQ ID NO: 16), TALEN nuclease, or zinc finger nuclease), or an IL-33 receptor (suppression of tumorigenicity (ST)2) inhibitor (such as, for example and antibody or small molecule inhibitor), or any combination thereof. In one aspect, the antifungal agent is administered prior to the onset of pancreatitis.

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer (including, but not limited to pancreatic ductal adenocarcinoma (PDAC)).

In one aspect, it is understood and herein contemplated that successful treatment of a cancer in a subject is important and doing so may include the administration of additional treatments. Thus, the disclosed methods of treating, reducing, inhibiting, decreasing, ameliorating and/or preventing a cancer and/or metastasis can include or further include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular IL-33 inhibitor, IL-33 receptor (ST2) inhibitor, MEK inhibitor, and/or anti-fungal agent is disclosed and discussed and a number of modifications that can be made to a number of molecules including the IL-33 inhibitor, IL-33 receptor (ST2) inhibitor, MEK inhibitor, and/or anti-fungal agent are discussed, specifically contemplated is each and every combination and permutation of IL-33 inhibitor, IL-33 receptor (ST2) inhibitor, MEK inhibitor, and/or anti-fungal agent and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 1. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with IL-33 or the IL-33 receptor (suppression of tumorigenicity (ST)2) such that IL-33 is inhibited from interacting with ST2 or ST2 is inhibited from signaling. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, VHH, and the like, including hybrid fragments. Thus, fragments of the antibodies that retain the ability to bind their specific antigens are provided. For example, fragments of antibodies which maintain IL-33 or ST2 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti IL-33 and/or anti-ST2 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable..

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### a) Results

### (1) Type 2 Immune Cell Infiltration Increases Significantly in PDAC Tumor Microenvironment

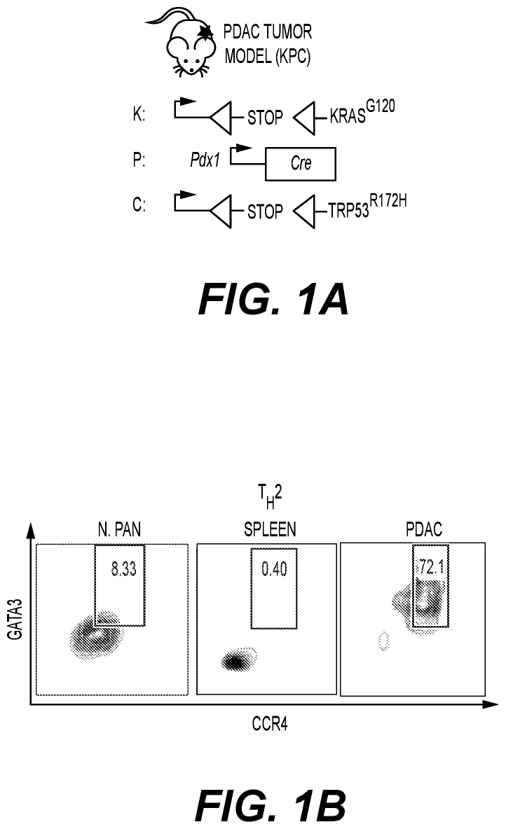

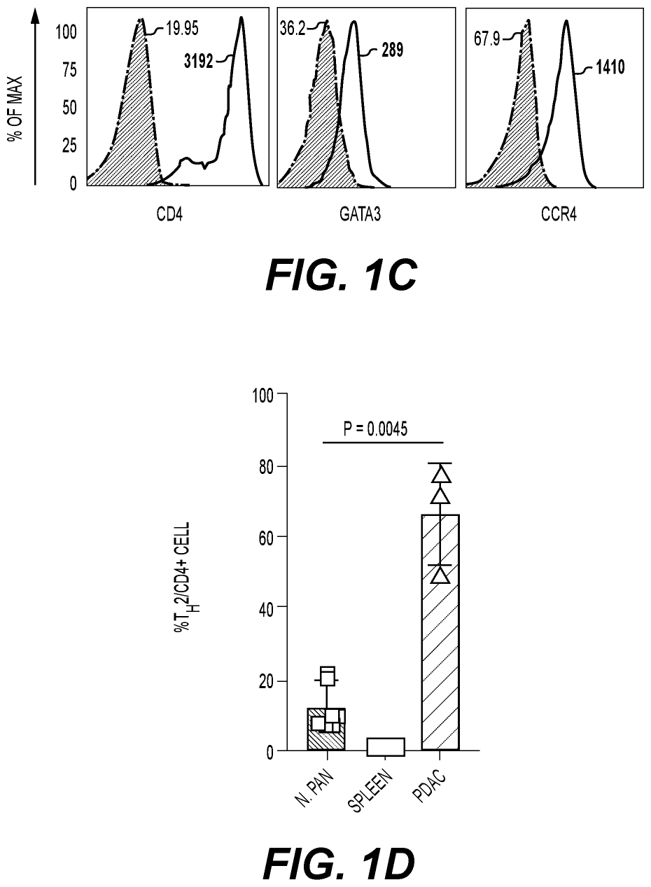

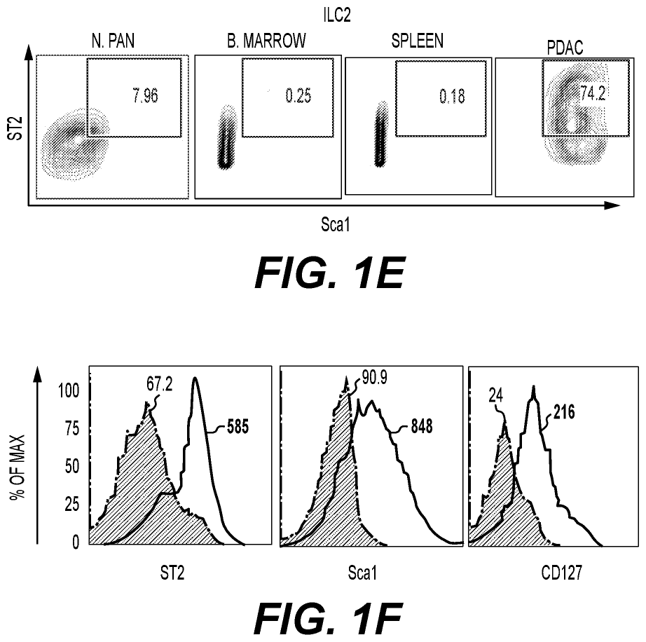

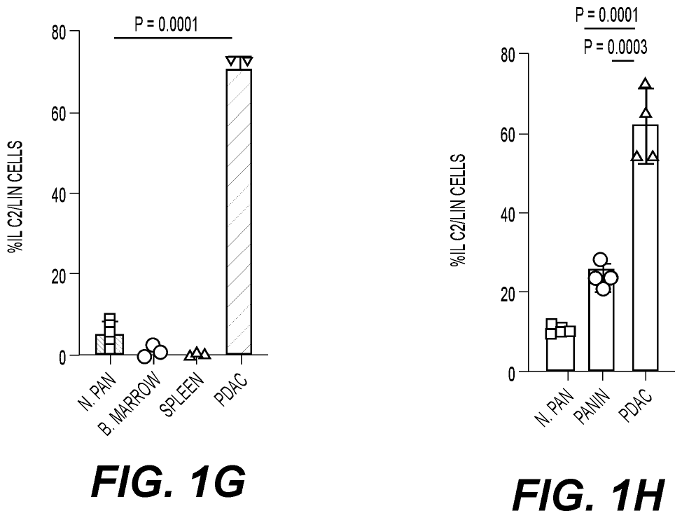

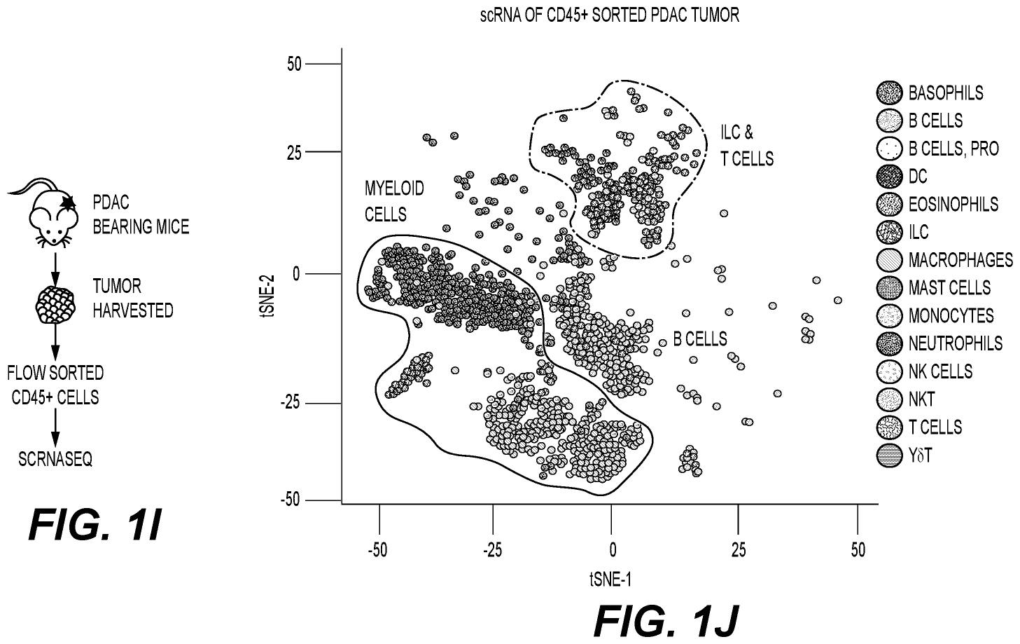

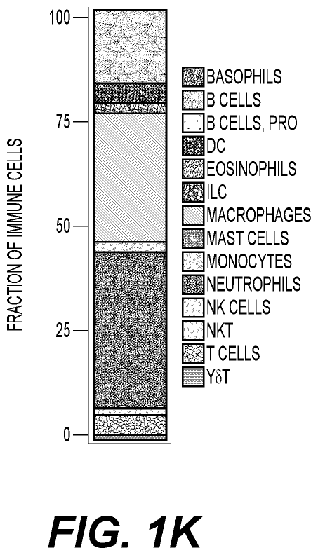

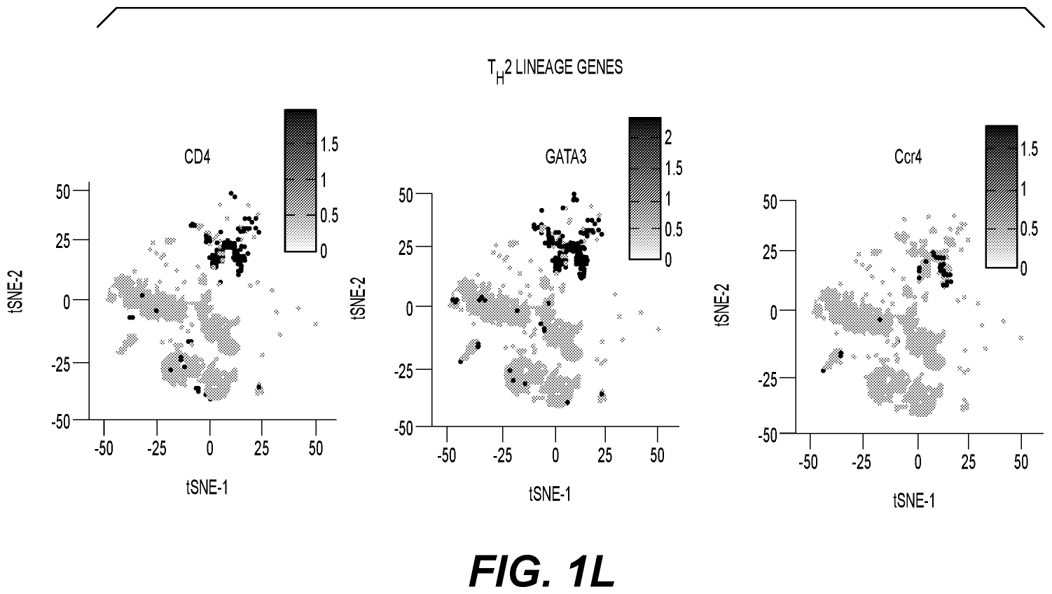

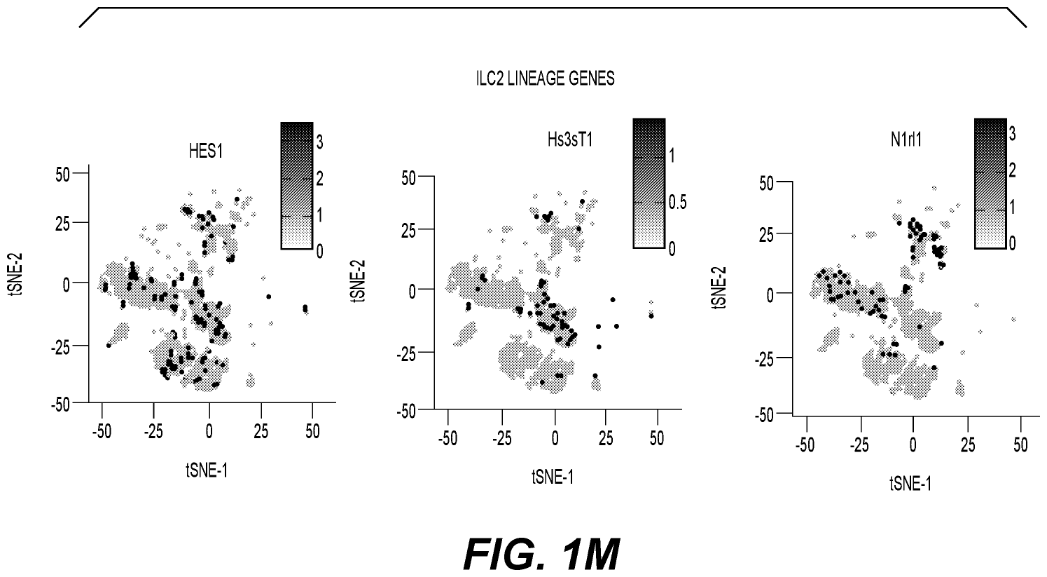

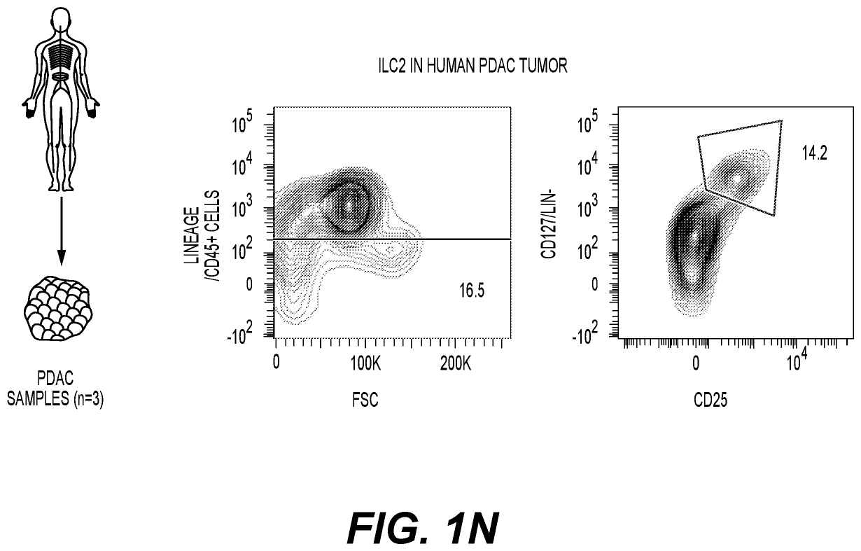

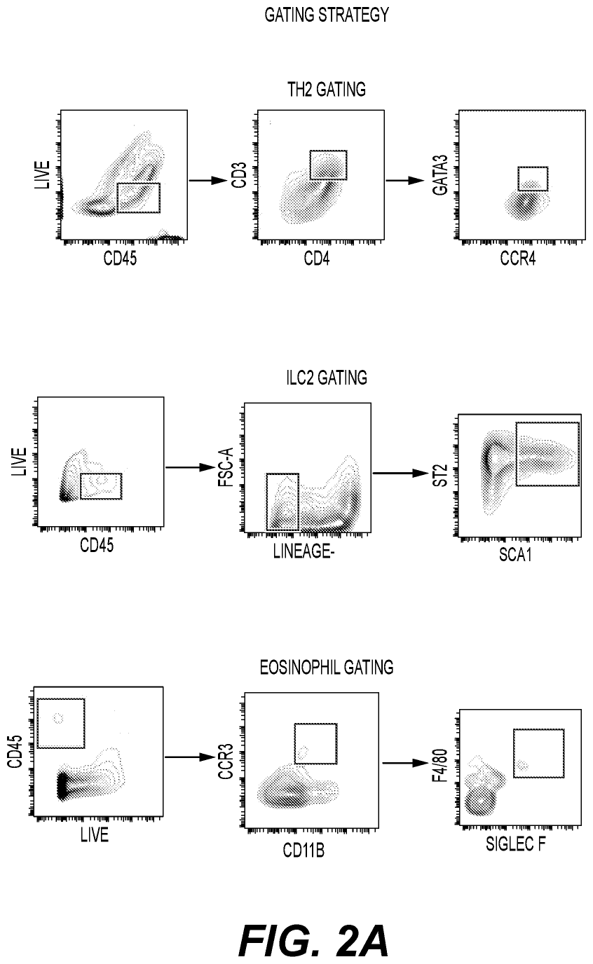

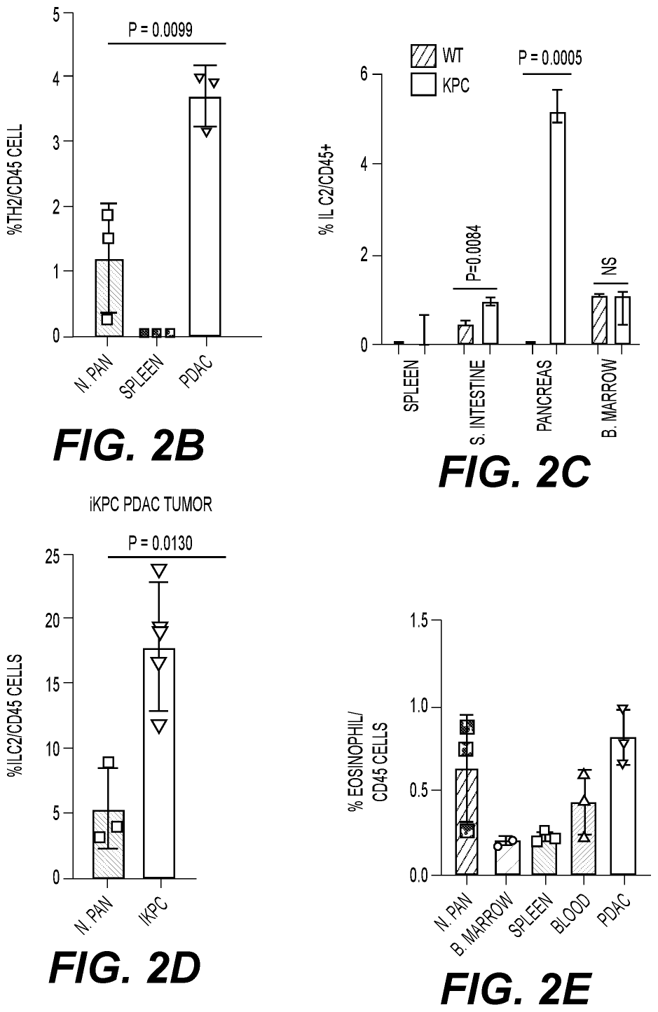

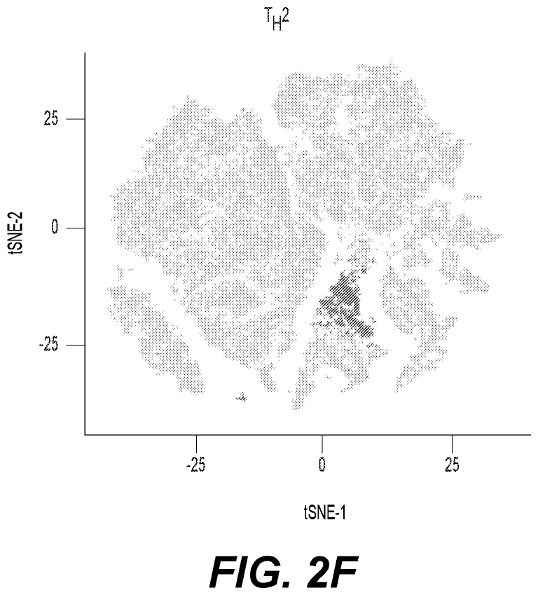

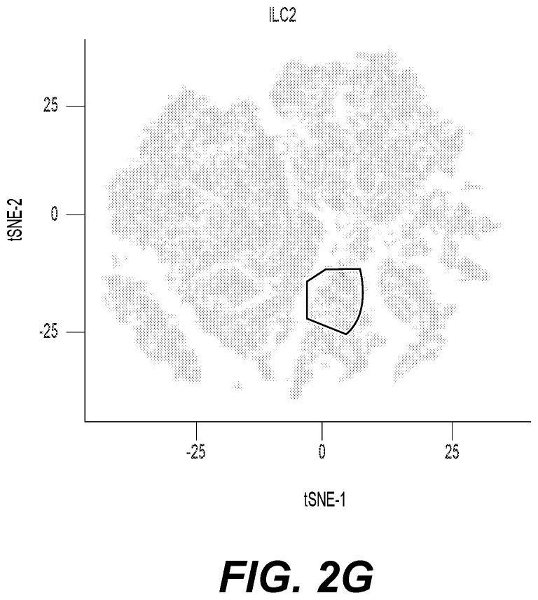

The observations that CD4⁺Gata3⁺ T_{H}2 cells infiltrate the TME and promote tumorigenesis via IL-4 and IL-13 prompted a more thorough characterization of the type 2 immunocytes present within the PDAC tumors of KPC (Kras^{G12D};Trp53^{R72H};Pdx-Cre) mice (FIG. 1A). Flow cytometry (FIG. 2A) revealed a dramatic expansion of T_{H}2 (CD4⁺Gata3⁺CCR4⁺) cells within the CD4⁺ T cell population in the PDAC TME (72.1%) compared to the normal pancreas (8.33%) and spleen (0.4%) (FIG. 1B-D, FIG. 2B). This was accompanied by a significant increase in ILC2 cells (Lin⁻Sca1⁺ST2⁺) in the PDAC TME (74.2%) compared to the normal pancreas (7.96%), spleen (0.18%) and bone marrow (0.25%) (FIG. 1E-G, FIG. 2C). Specifically, the frequency of ILC2 cells was approximately 60% of Lin cells in the PDAC (KPC model) compared to <10% in the normal pancreas and ˜25% in PanIN (FIG. 1H). Similarly, the frequency of ILC2 cells was approximately 45% of Lin cells in the PDAC (iKPC model) compared to <10% in the normal pancreas (FIG. 2D).

To solidify our flow-based immunophenotyping data, we used single-cell RNA-seq (scRNA-seq) analysis to identify the presence of the type 2 immunocytes in the PDAC TME. We sort-purified CD45⁺ cells and used the 10× Genomics platform for scRNA-seq of the immune populations in the PDAC tumor samples (FIG. 1I-J). The majority of immune cell populations identified were myeloid cells (macrophages, monocytes, DCs and neutrophils: ˜74.72%), followed by lymphoid populations (16.45%) (FIG. 1K, FIG. 2E). Using previously reported gene signatures for T_{H}2 and ILC2, we found that the T_{H}2 cluster was enriched for Cd4, Gata3 and Ccr4 genes (FIG. 1L, FIG. 2F) and ILC2 clusters were enriched for Hes1, Hs3st1 and Il1rl1 genes (FIG. 1M, FIG. 2G), which are bonafide markers of T_{H}2 and ILC2 cells, respectively. Finally, we analyzed fresh human PDAC samples by flow cytometry and found that ILC2 cells accounted for 14.2% of the Lin⁻ cells (FIG. 1N). Overall, these results show that the murine and human PDAC TME contain abundant T_{H}2 and ILC2s cells.

### (2) IL33 is a Downstream Target of Oncogenic Kras*

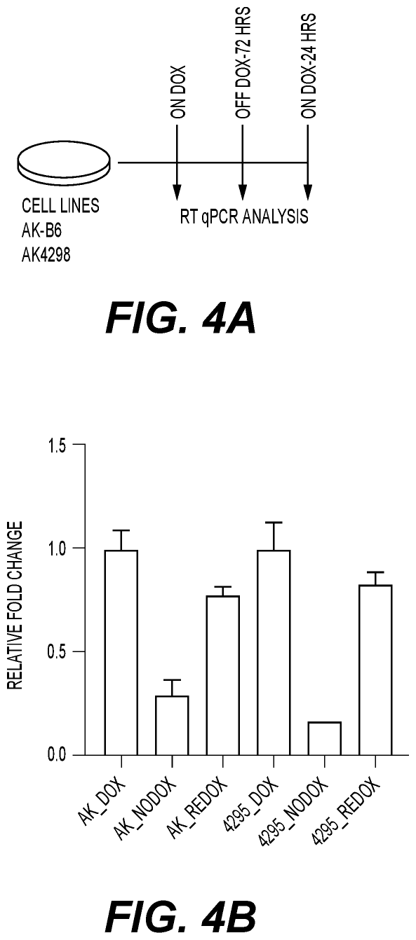

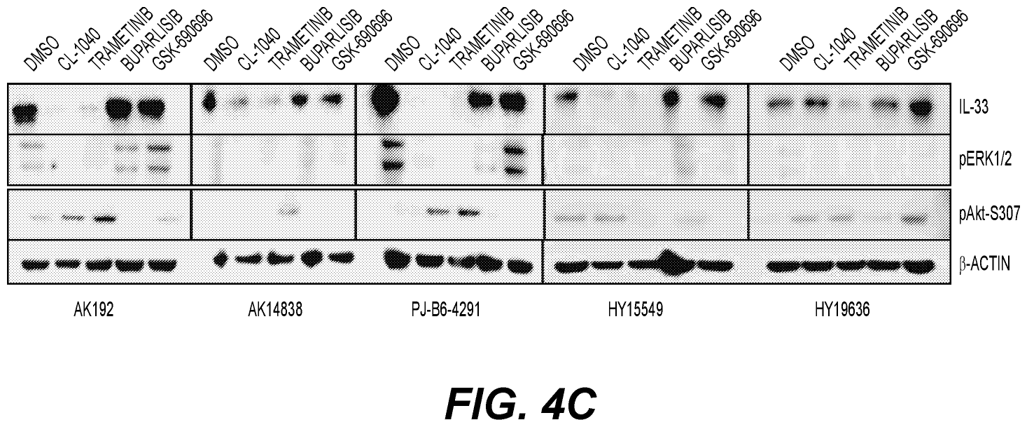

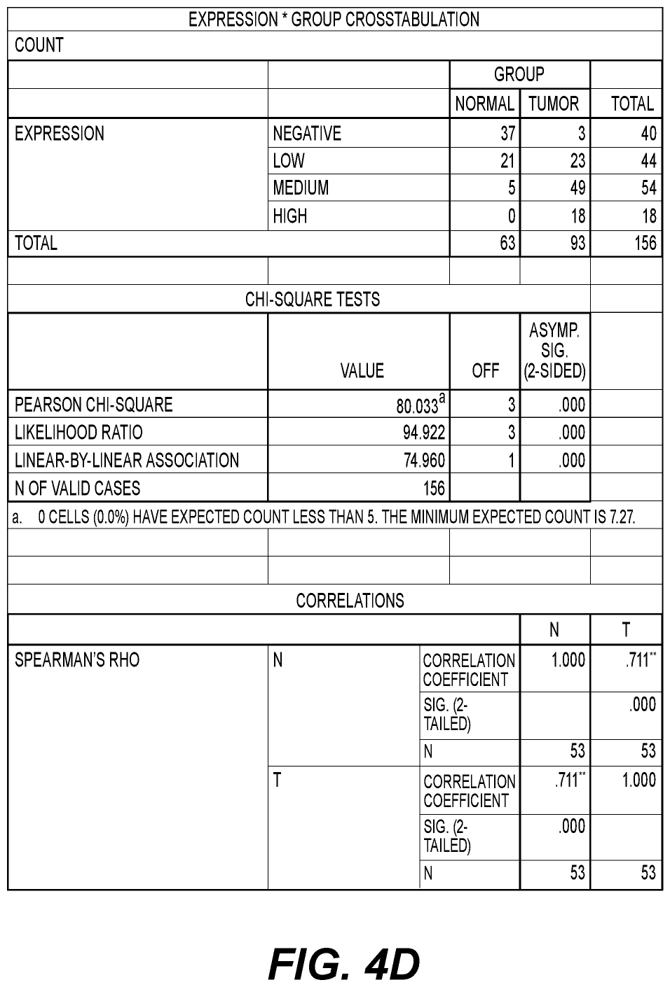

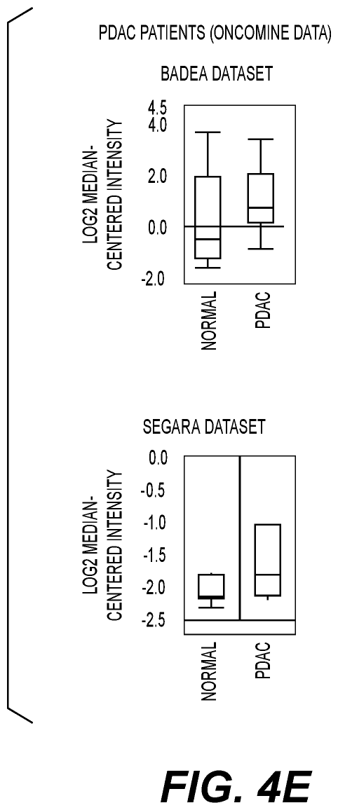

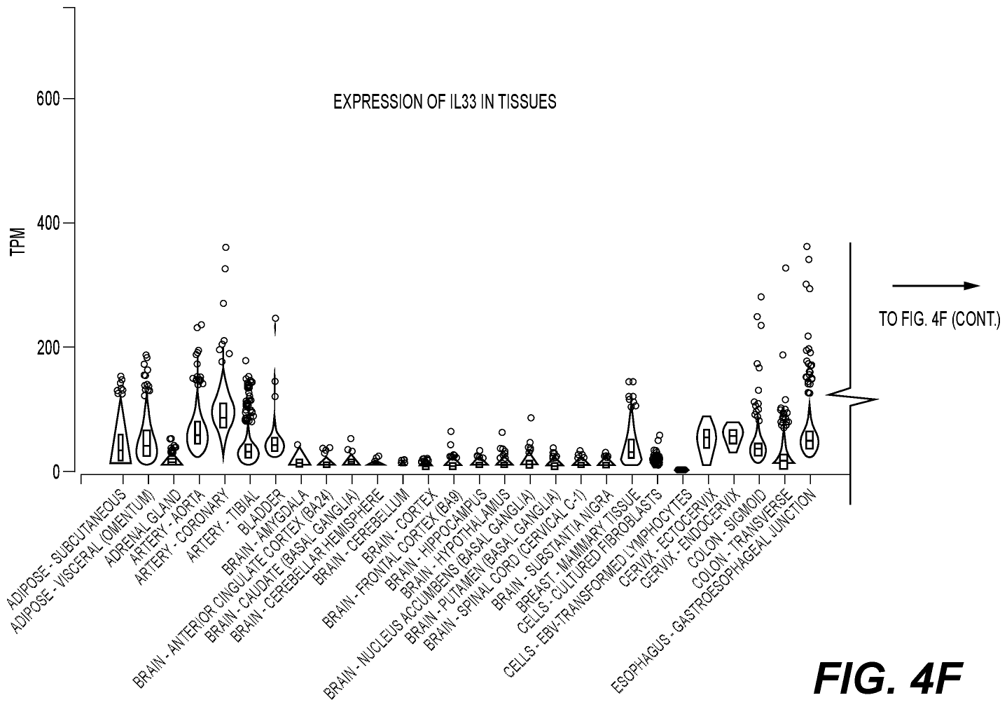

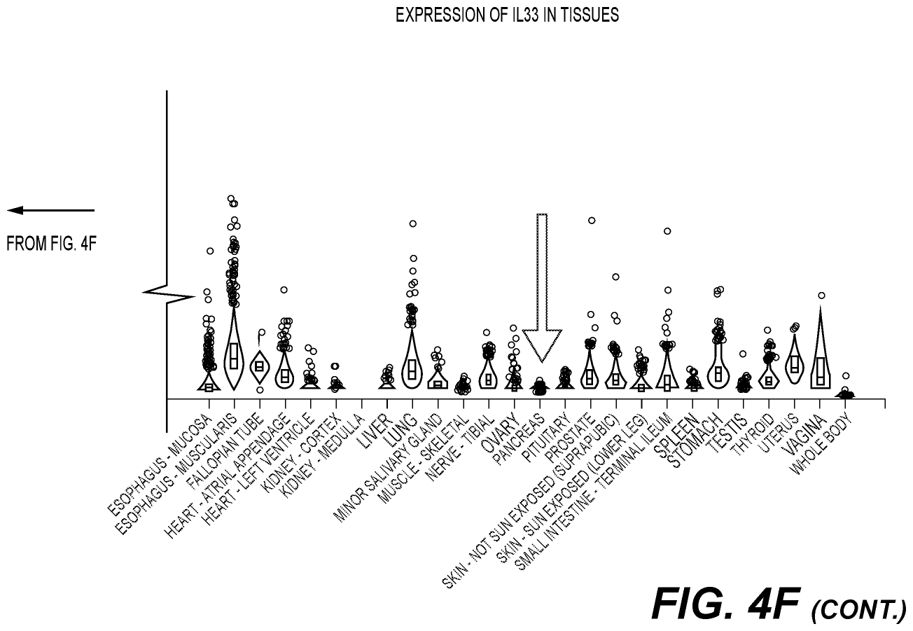

Type 2 immunocytes are detected in the early stages of PDAC tumorigenesis, prompting speculation that a chemotactic factor secreted by cancer cells can recruit and activate type 2 immunocytes. Given that KRAS mutation is an early genetic alteration and drives tumor initiation, we examined the Kras*-regulated transcript of multiple PDAC cell lines derived from the Kras* inducible model of PDAC (iKPC: LSL-tet-O-Kras^{G12D}; LSL-p53^{+/-}; p48-Cre;LSL-Rosa26-rtTA) (FIG. 4A). RNAseq analysis of iKPC cell lines included Kras* ON (doxycycline ON), Kras* OFF 2 days and Kras* OFF 4 days. Among the top cytokine genes that are regulated by Kras* and enriched in the KRAS signaling network is the alarmin gene, IL33 (FIG. 4B-D). IL33 expression is ˜30-fold higher in Kras* ON compared to Kras* OFF samples, as validated by quantitative real-time PCR (qRT-PCR) analysis (FIG. 4E).

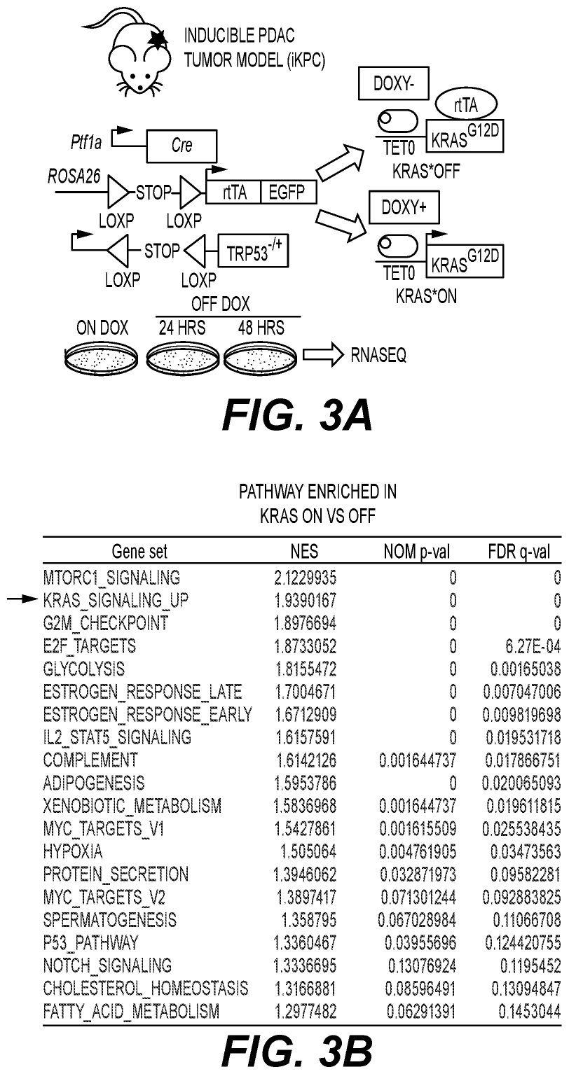

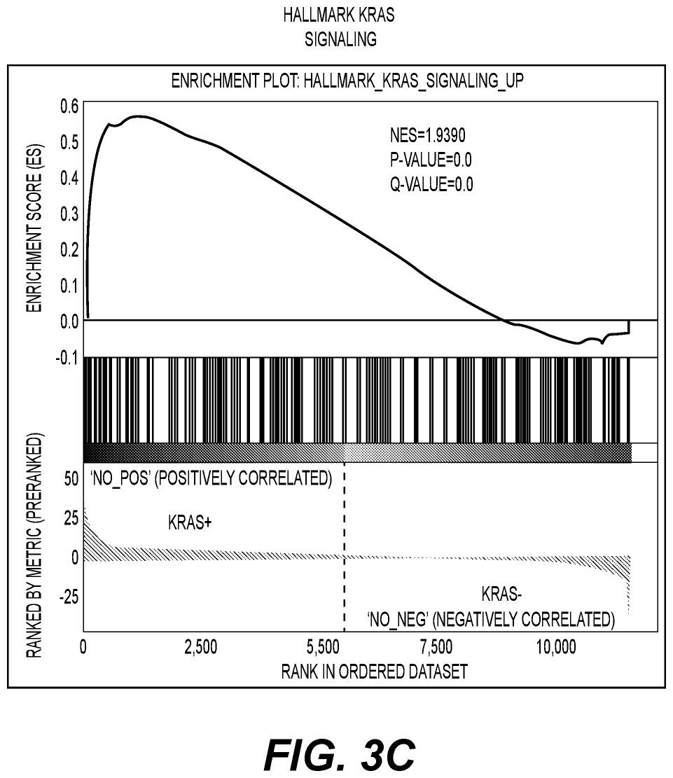

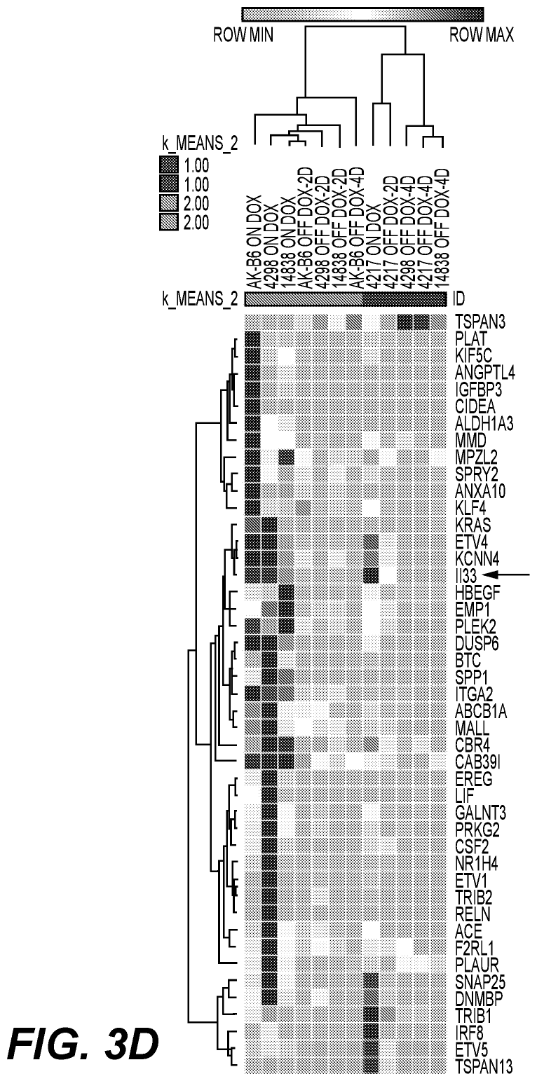

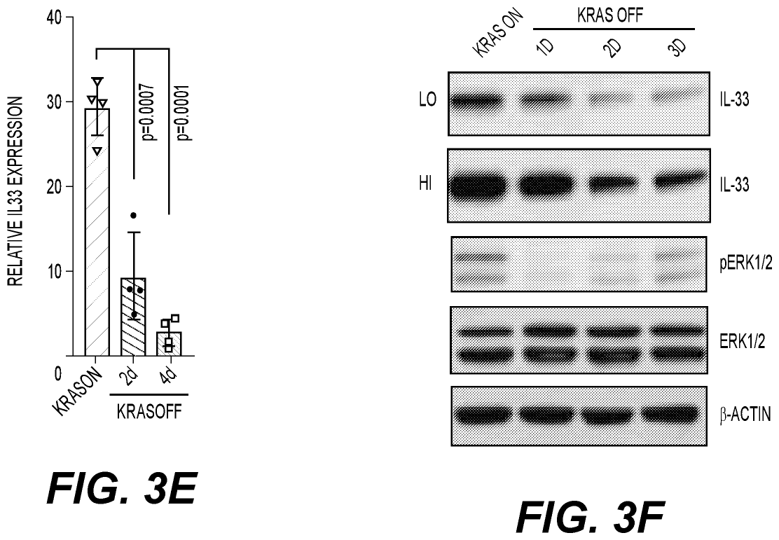

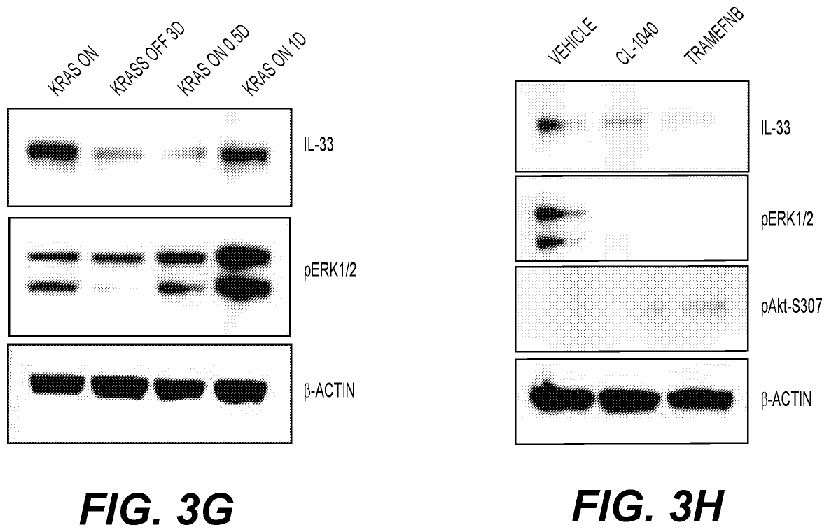

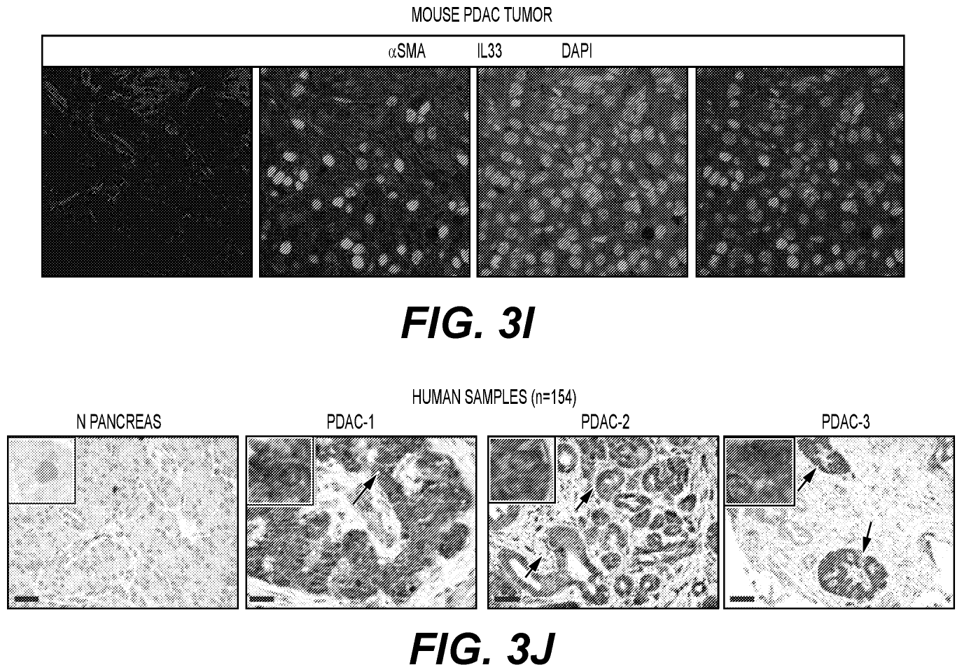

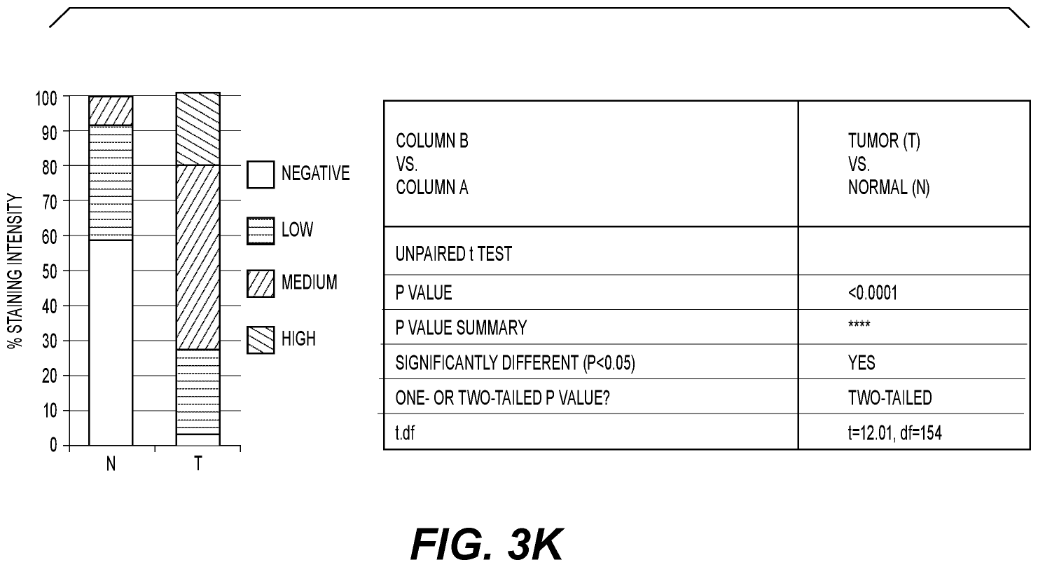

The above in silico findings were further validated by western blot analysis showing a complete loss of IL33 expression upon abolishing Kras* signaling (FIG. 3F-G, FIG. 4A-B). We further validated this finding using cell lines derived from KPC tumor model of PDAC (FIG. 1A). Treatment with inhibitors (CI-1040, Trametinib) of MEK, a downstream target of Kras signaling, resulted in complete inhibition of IL33 expression in these cell lines (FIG. 3H, FIG. 4C). However, treatment with PI3K inhibitors (Buparlisib and GSK-690696) did not lead to a reduction in IL33 expression, indicating that IL33 is induced via a Kras-ERK signaling pathway independent of PI3K (FIG. 4C).

We next evaluated IL33 expression in the KPC mouse model and in human PDAC tissue. In the KPC model, IL33 expression was mostly observed in the cancer cells with nominal expression observed in fibroblasts or immune cells (FIG. 3I). Further, these murine data align with the human tumor data showing that IL33 expression was increased in patients, with ˜20% PDAC patients showing high and ˜52% showing moderate IL33 expression (FIGS. 3J and 3K). Similar to mice, IL33 expression in human PDAC specimens was mostly restricted to cancer cells with minimal staining in the stromal compartment (FIG. 3J, FIG. 4D-E). The normal pancreas did not express detectable IL33 (FIG. 3J, FIG. 4F). These data show that Kras*-MEK signaling in tumor cells can induce IL33 and that cancer cells are the major source of IL33 in the PDAC TME.

### (3) IL33 Expression is Required for the Recruitment of Type 2 Immunocytes

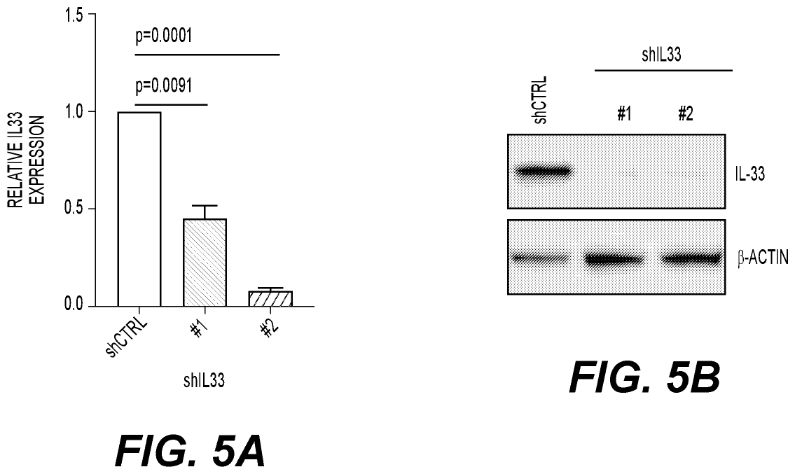

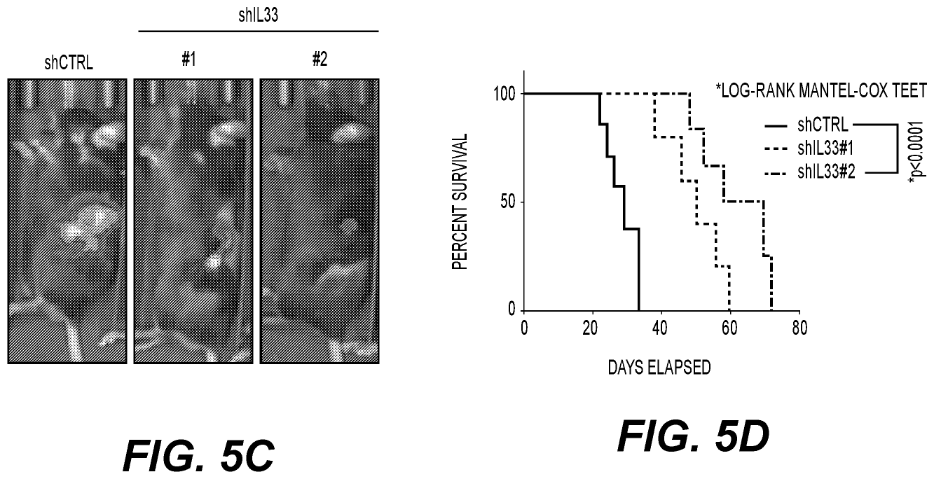

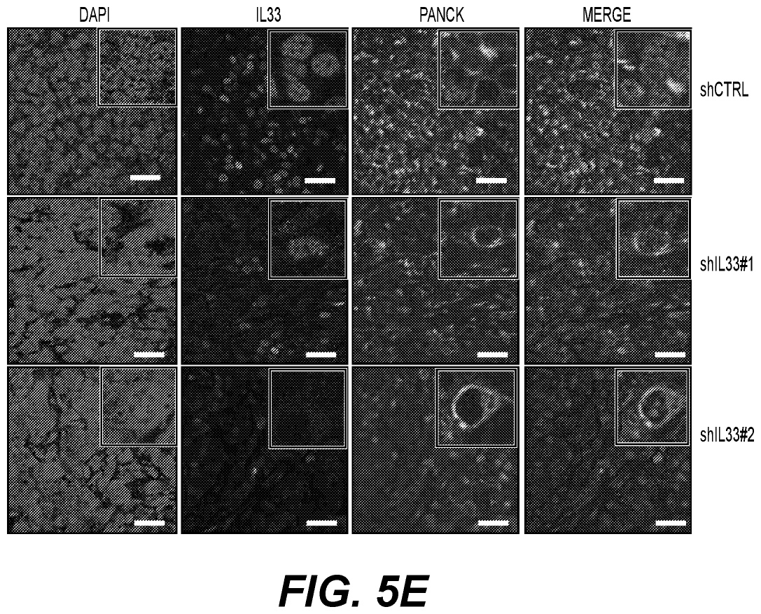

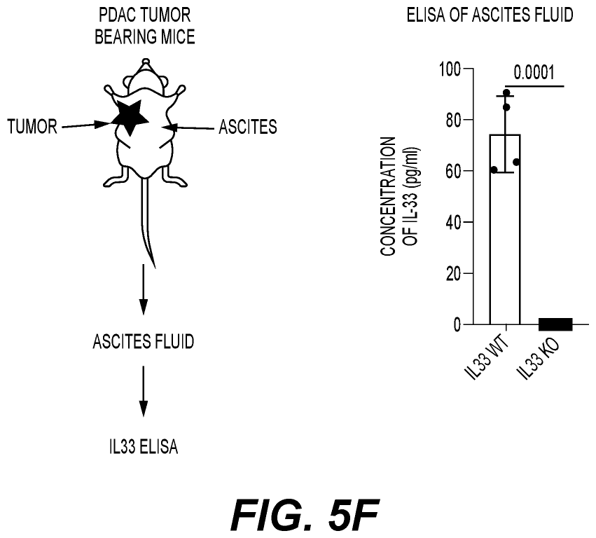

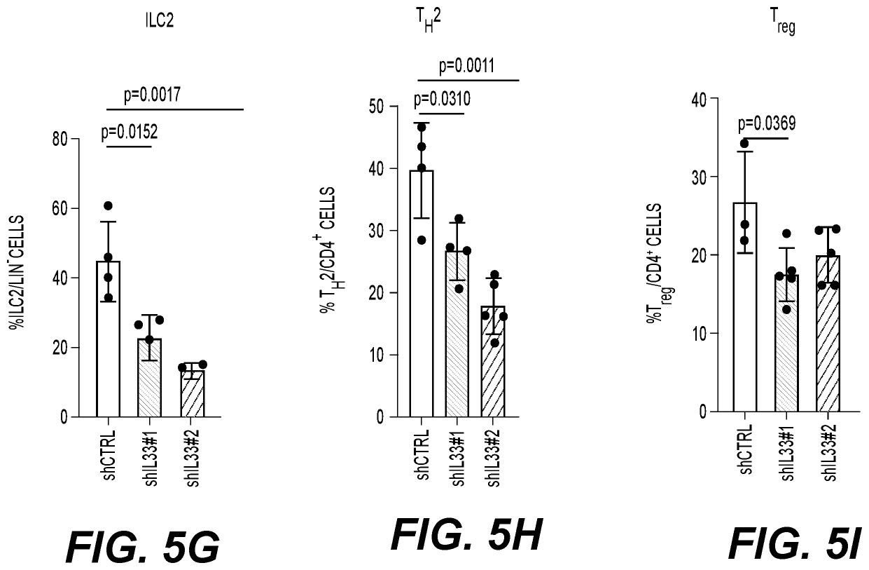

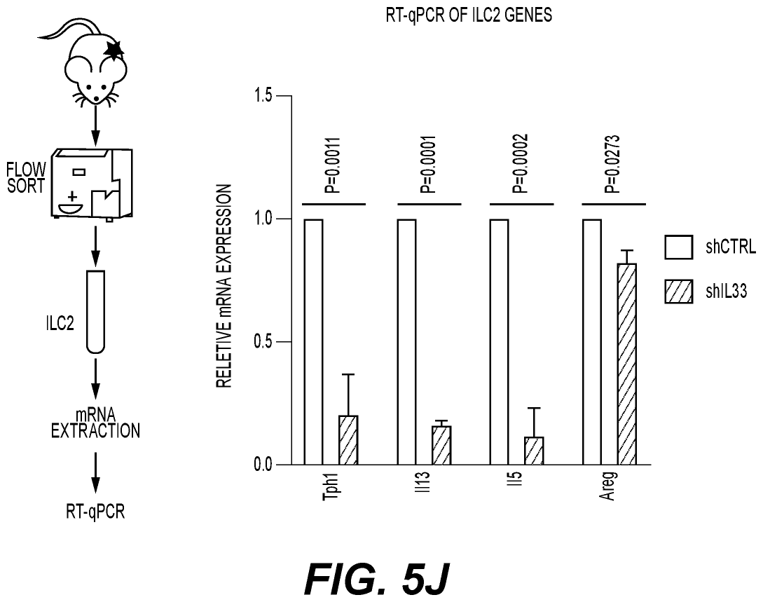

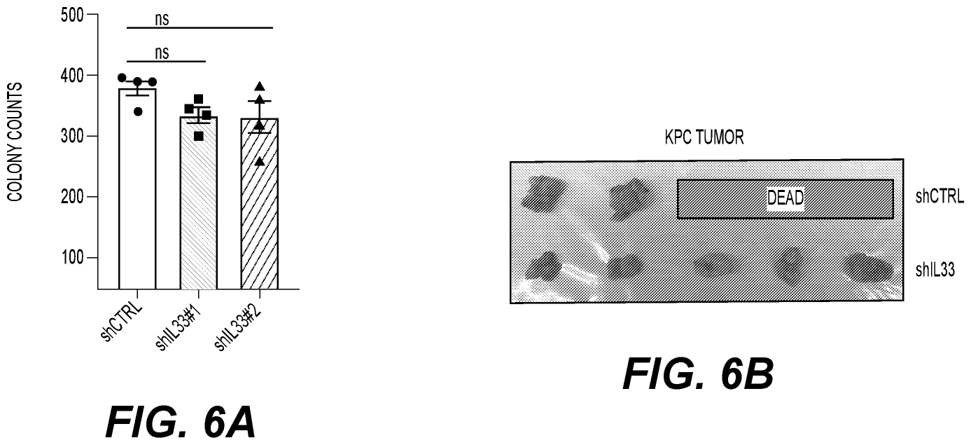

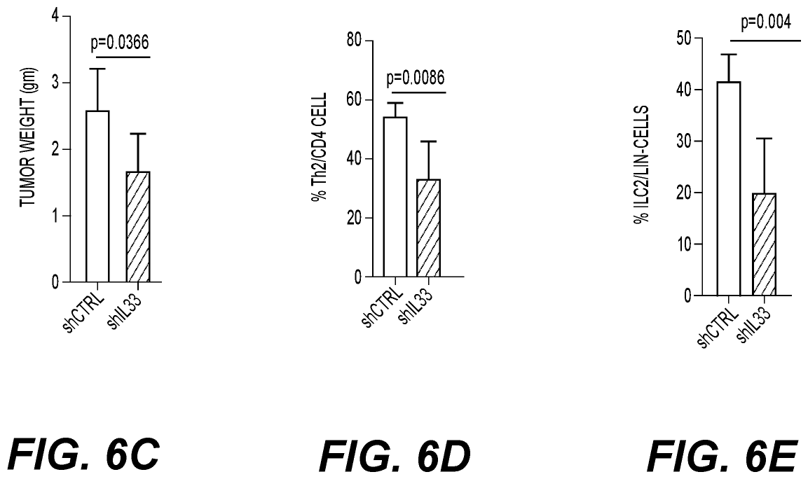

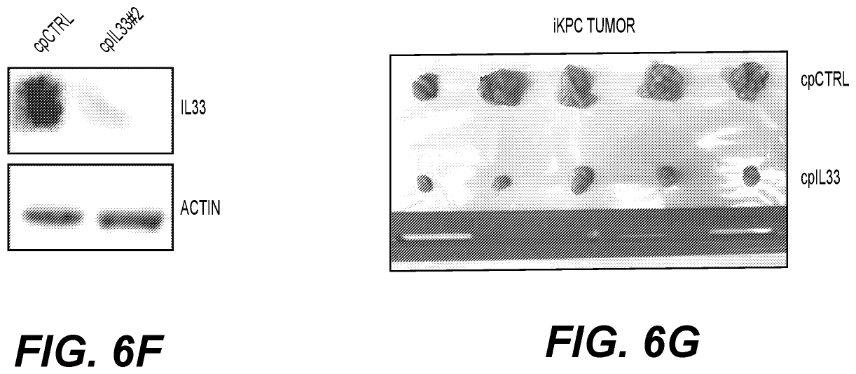

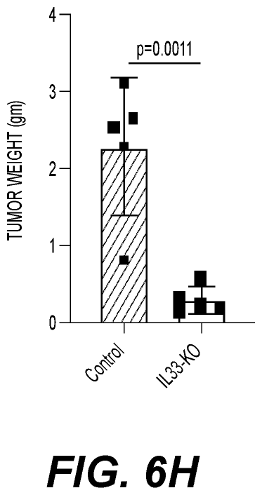

The T_{H}2 and ILC2 infiltration increases significantly during PDAC tumorigenesis. To test the requirement of IL33 expression by cancer cells to recruit type 2 immunocytes, IL33 was depleted in cancer cells by lentivirus transduction of small hairpin (sh)RNA (FIG. 5A-B). While shRNA-mediated depletion of IL33 had no effect on cell growth in vitro (FIG. 6A), reduced IL33 levels resulted reduced tumor burden and increased survival in a syngeneic orthotopic model of PDAC (FIG. 5C-D, FIG. 6B-C). Immunofluorescence of these tumors confirmed that PDAC cancer cells are the major source of IL33 and that the IL33 signal is diminished by shRNA-mediated depletion (FIG. 5E). Moreover, malignant ascites, observed in ˜20% of PDAC patients, is also a feature of the PDAC mouse model. In tumor-bearing mice, IL33 depletion in cancer cells led to a decreased IL33 protein levels in the ascites fluid indicating a reduced production of IL33 by the cancer cells (FIG. 5F).

To establish the mechanistic link between IL33 secretion and ILC2 trafficking flow-cytometry analysis of the TME show that IL33 depletion reduced both T_{H}2 and ILC2 infiltration, supporting the role of cancer cell-derived IL33 in inducing type 2 immune responses in the TME (FIG. 5G-H, FIG. 6D-E). In addition to ILC2 and T_{H}2 cells, T_{reg} cells are known to express IL33 receptors (ST2) and can respond to IL33 signaling. Accordingly, IL33 depletion resulted in a small but significant reduction in T_{reg} infiltration in the TME (FIG. 5I). The above shRNA depletion findings mirrored those from CRISPR-Cas9 knockout of IL33 which showed a strong reduction in T_{H}2 infiltration (FIG. 6F-H). In addition, IL33 knockout produced reductions in other IL33 responding immunocytes, such as MDSCs and T_{regs} and no significant change in CD8⁺ T cells, neutrophils and B cell population (FIG. 6I). Notably, in addition to the diminished infiltration of ILC2 cells in IL33-deficient tumors, the activation marker required for ILC2 function, Tph1 and the effector cytokines, IL13, IL5 and Areg mRNA levels were also reduced, which indicates that small population of resident ILC2 cells present within the PDAC TME are functionally inactive with decreased IL33 (FIG. 5J). Together, these data established that cancer cell-derived IL33 recruits and activates type 2 immune cells into the PDAC TME.

(4) Intratumor Fungi Facilitate the Release of IL33 from PDAC Cells

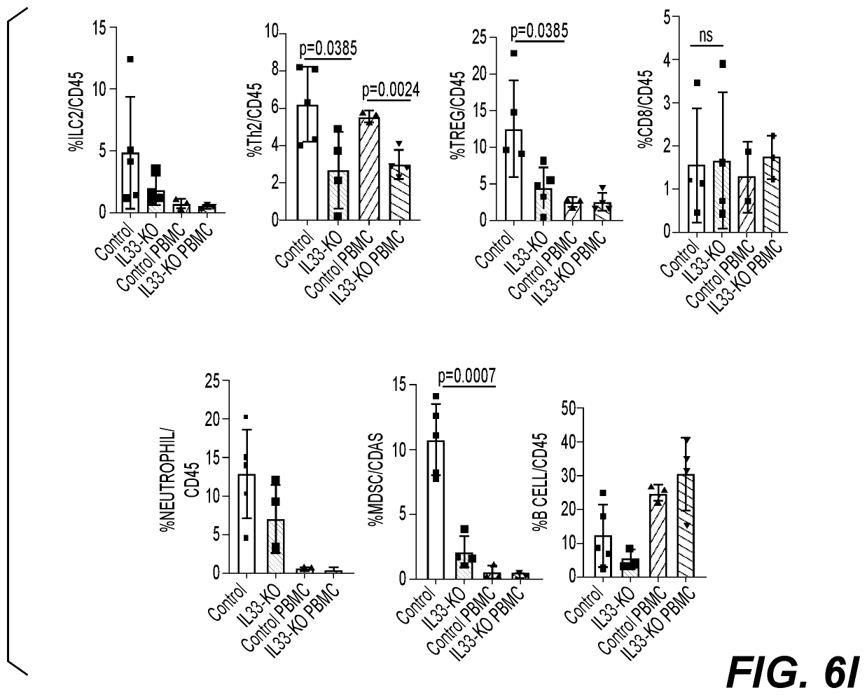

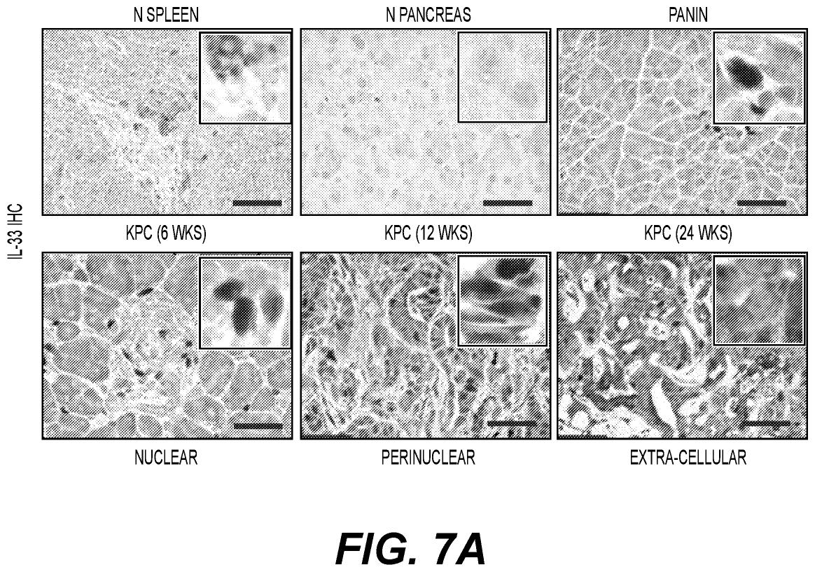

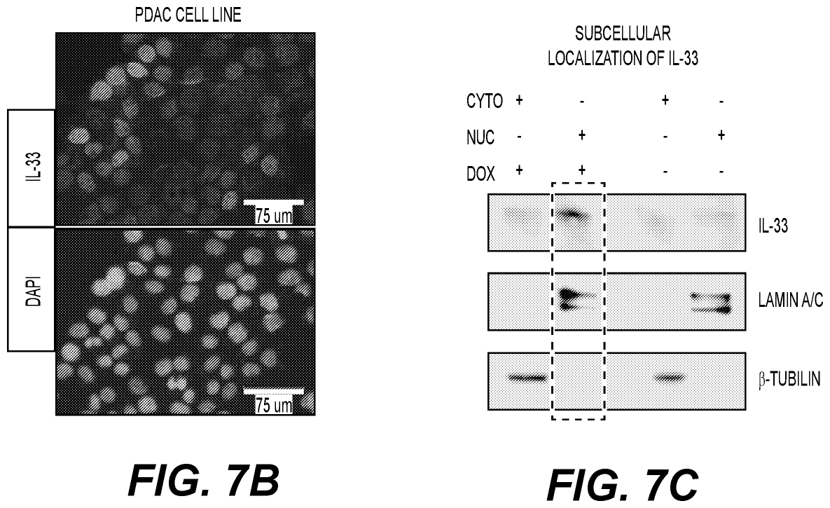

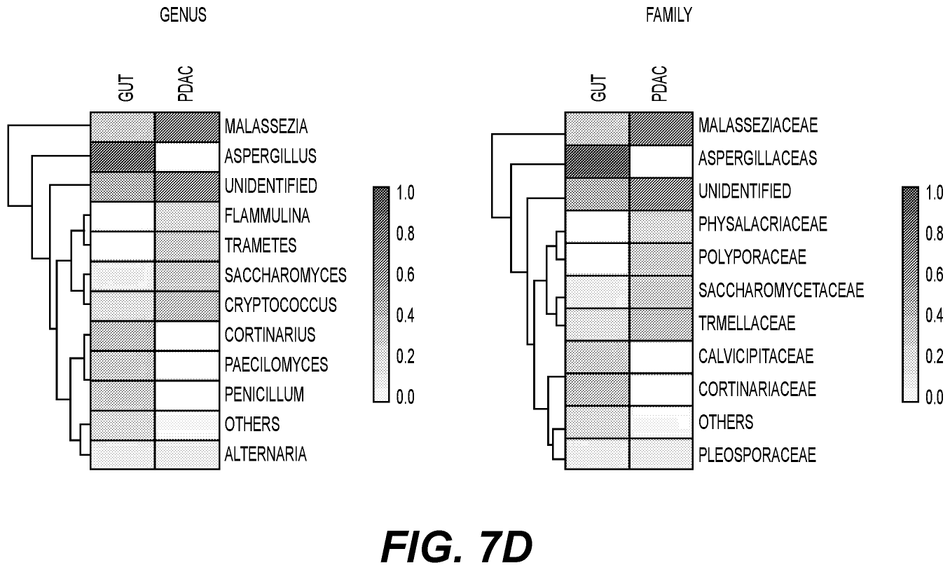

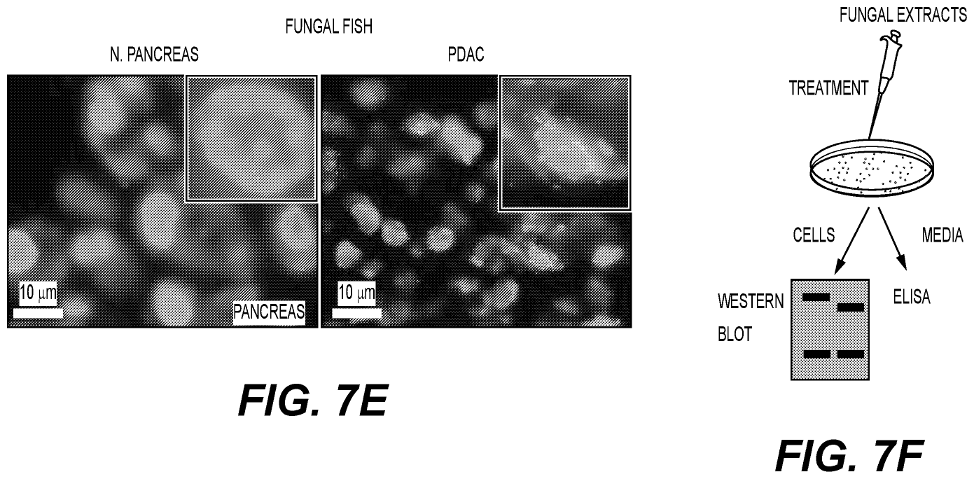

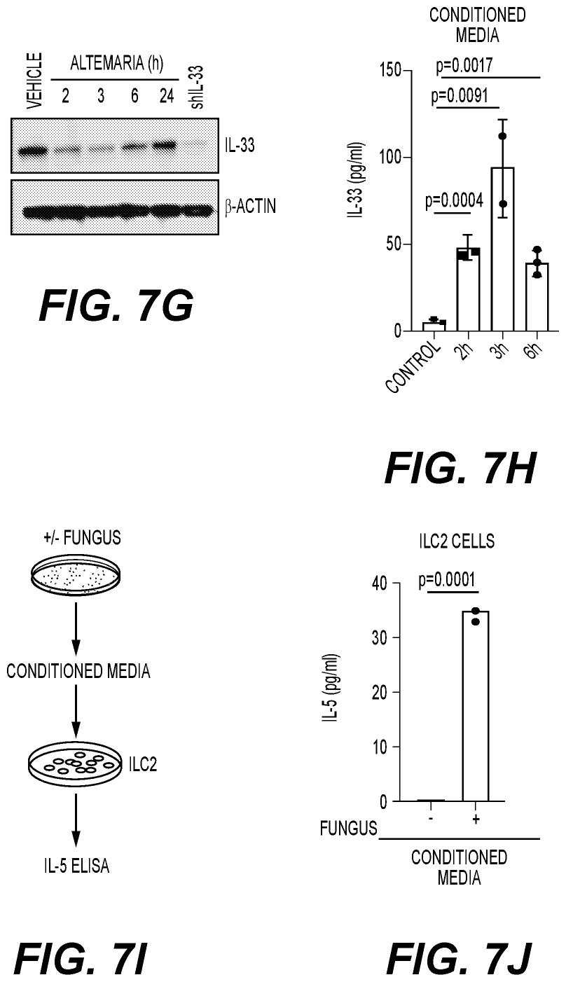

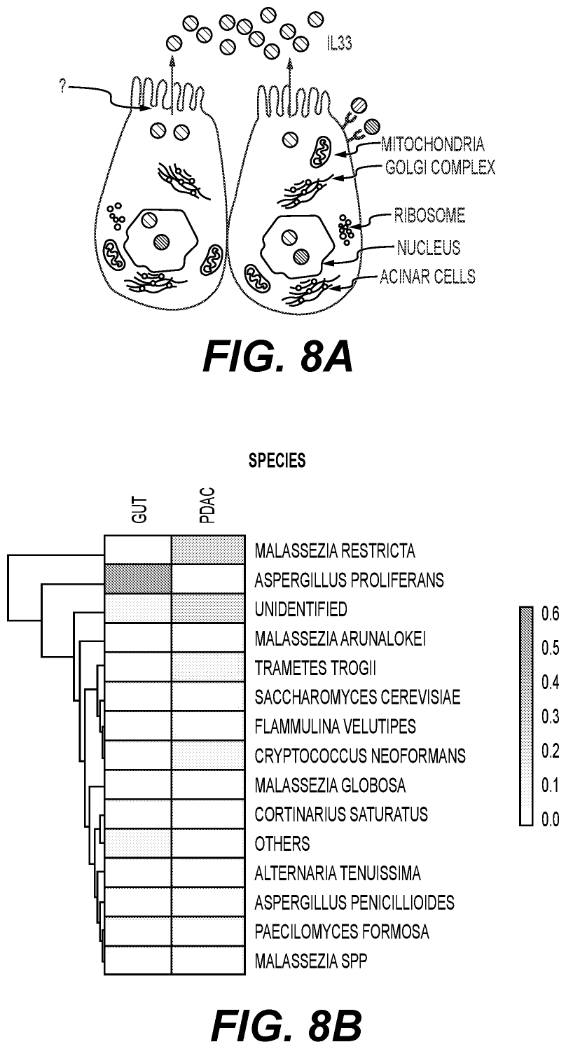

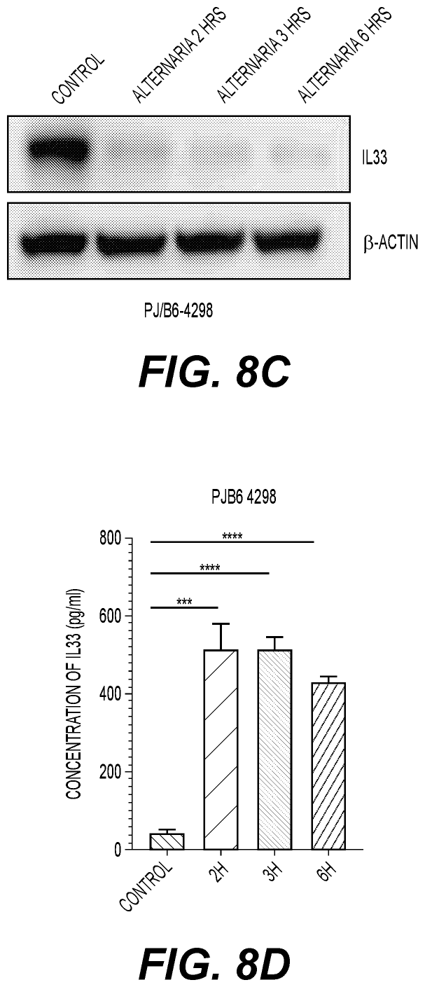

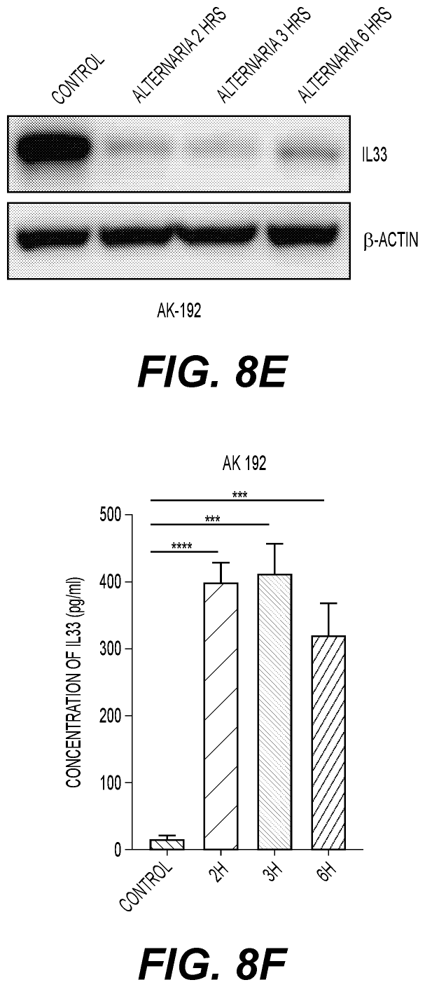

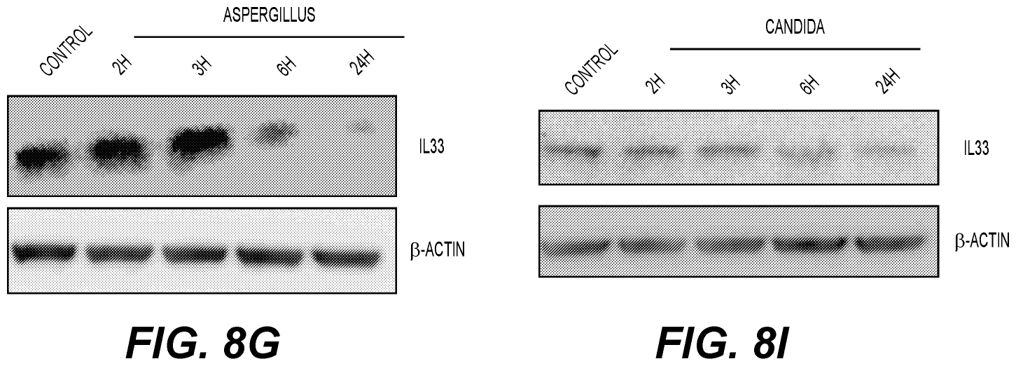

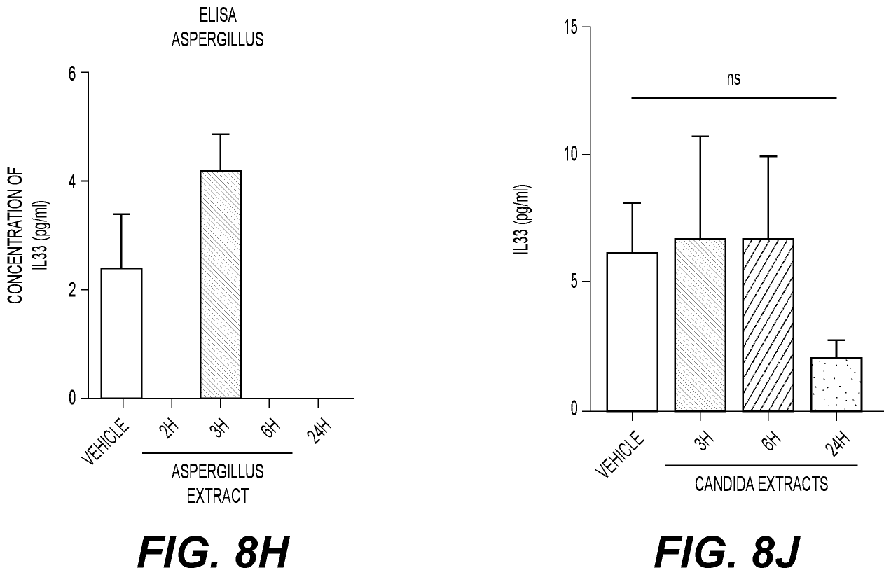

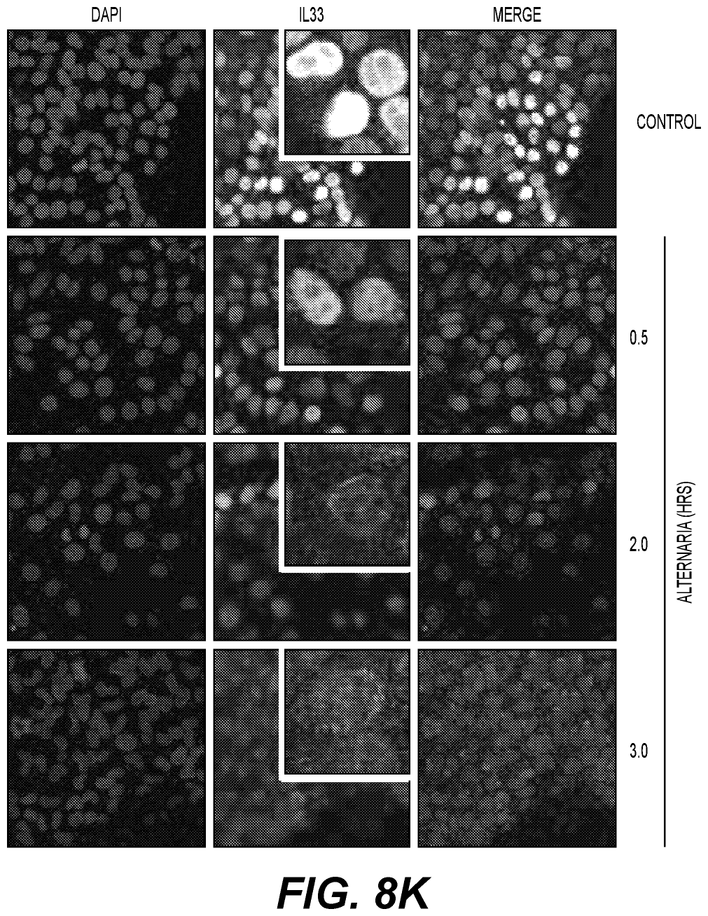

Immunohistochemistry analysis of PDAC tumors revealed that IL33 expression and localization track tumor progression, with IL33 expression being predominantly nuclear in the preneoplastic stages (PanIN1-3) compared to advanced adenocarcinoma when its expression becomes wide-spread across the cytosol and extracellular milieu (6 vs. 24 week-old PDAC tumors) (FIG. 7A). In contrast, immunofluorescence staining of IL33 of PDAC cells in culture shows staining primarily in the nucleus of PDAC cells (FIG. 7B), confirmed further by subcellular fractionation studies (FIG. 7C). These observations prompted consideration of the possibility that the secretion of IL33 is regulated by pathways and factors beyond Kras* signaling that are operative in the TME (FIG. 8A). The notion that IL33 secretion requires an environmental stimulus is further fueled by the fact that IL33 is a DAMP molecule and its extracellular secretion is tightly regulated in normal cells to avoid unwanted immune responses.

Studies in a murine allergic model show that IL33 can be secreted by lung epithelial cells in the presence of fungus, thereby exacerbating allergic responses. Interestingly, recent studies have reported that the intra-tumoral microbiome significantly increases in PDAC and plays an essential role in PDAC tumorigenesis. Specifically, the fungal-biome (mycobiome) which is present in the gut lumen have been shown to migrate to the pancreas via the sphincter of Oddi to populate the TME. As a first step, 18S ribosomal RNA (rRNA) sequencing confirmed the presence of mycobiome in the PDAC mouse model (FIG. 7D, FIG. 8B). Fungal communities were detected in both tumor and gut samples of a PDAC-bearing mice and were present in much higher abundance in PDAC tumors. Distinct fungal species, Malassezia and Alternaria, were documented as the most abundant fungi in PDAC tumors. To confirm the 18S rRNA sequencing, we conducted fluorescence in situ hybridization (FISH) to probe for the presence of fungal DNA in the normal pancreas and PDAC tumor, confirming a higher fungal abundance in the PDAC tumor compared to the normal pancreas (FIG. 7E).

Next, in vitro assays were performed to ascertain the direct role of fungus on IL33 secretion. We used an array of fungi identified in the 18S rRNA sequencing, as well as those reported earlier in PDAC tumors. There is a time-course dependent loss of IL33 in PDAC cells treated with extracts of Alternaria alternata (FIG. 7F-G, FIG. 8C, E), as shown by western blot analysis. The secretion peaks at 3 hours post-fungal extract treatment and tapers off at 6 hours. The secretion completely ceases at about 24 hours post-treatment. Simultaneously, an ELISA detected IL33 in the spend media, which coincides with the loss of IL33 in cell lysates, (FIG. 7H, FIG. 8D, F). Interestingly, a similar experiment conducted with Aspergillus and Candida sp. did not yield similar results, indicating that specific fungi species regulate IL33 secretion in PDAC cancer cells (FIG. 8G-J). In a separate experiment, cancer cells treated with Alternaria alternata showed a time-course dependent loss of IL33 (FIG. 8K).

To determine whether the IL33 secreted into the spent media can activate ILC2 cells, we conducted a functional assay where we sort-purified ILC2s from mouse spleen and treated the ILC2s with the fungal-treated spent media from the PDAC cell line (FIG. 7I). An ELISA of IL5, an activation marker for ILC2, showed robust IL5 secretion by ILC2 cells (FIG. 7J).

### (5) Intratumor Fungi Accelerate PDAC Tumor Growth

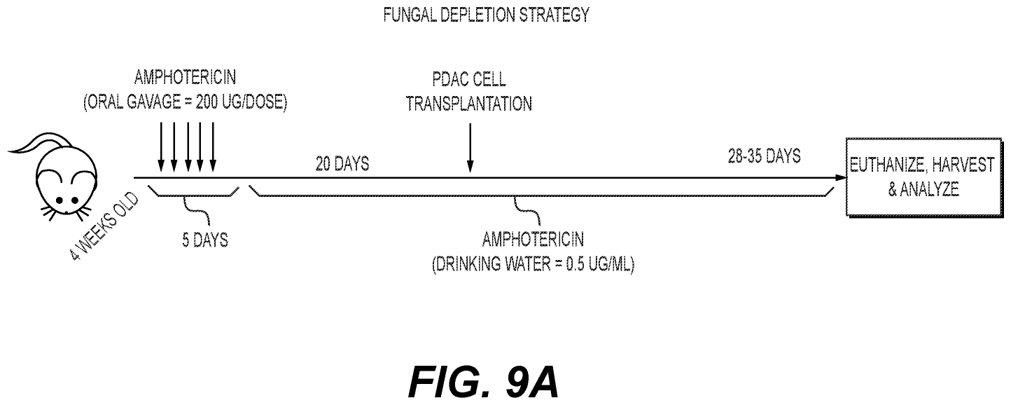

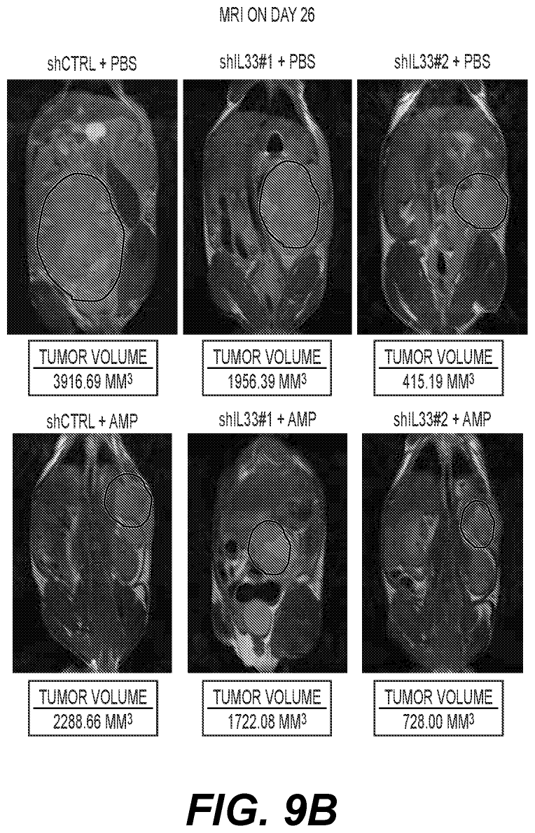

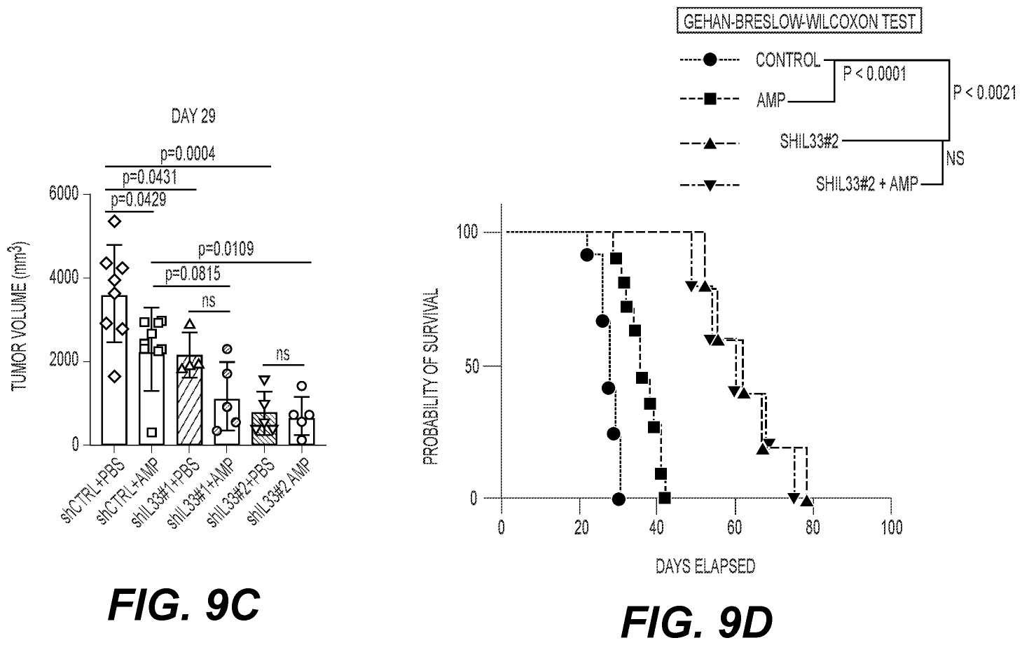

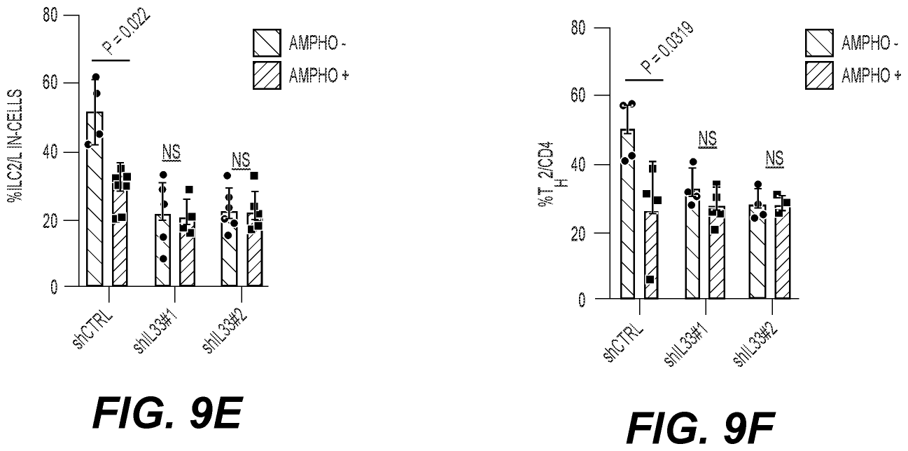

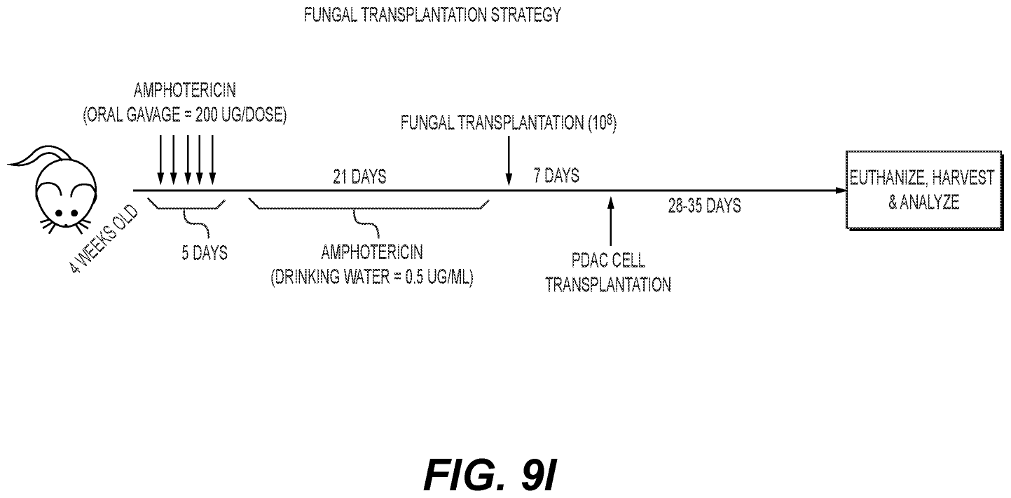

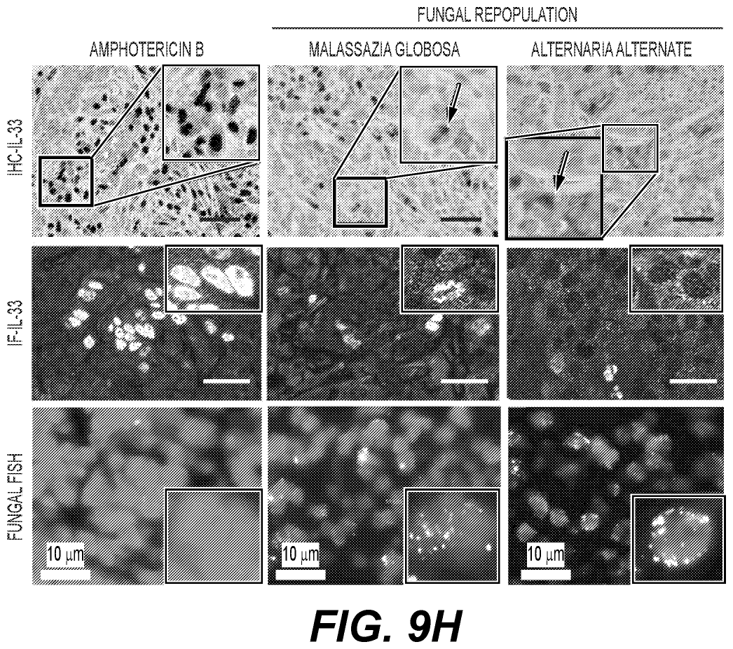

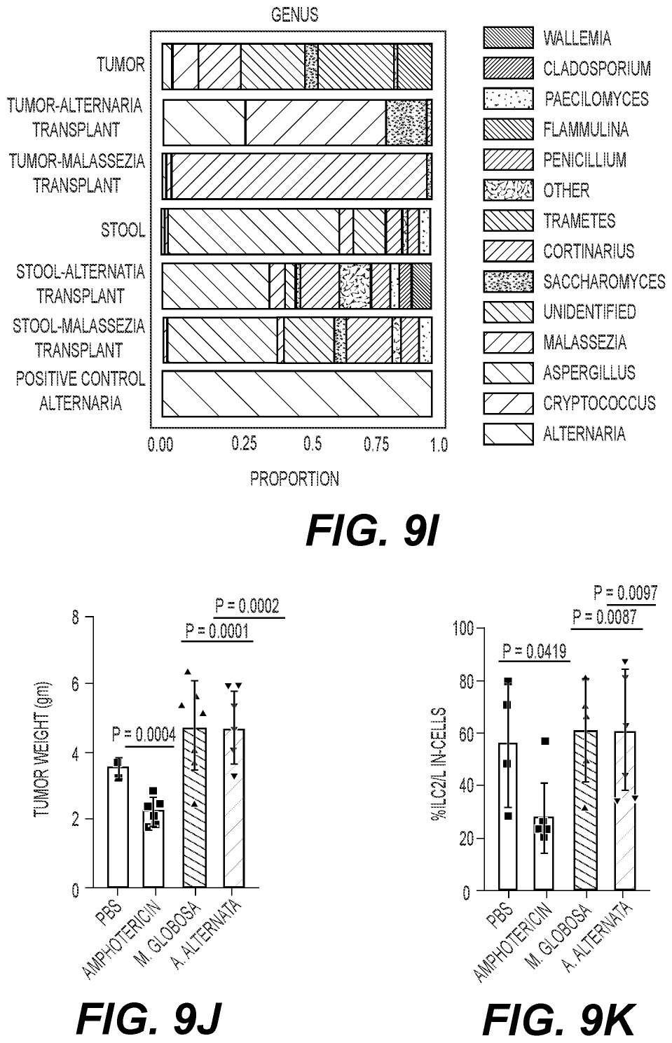

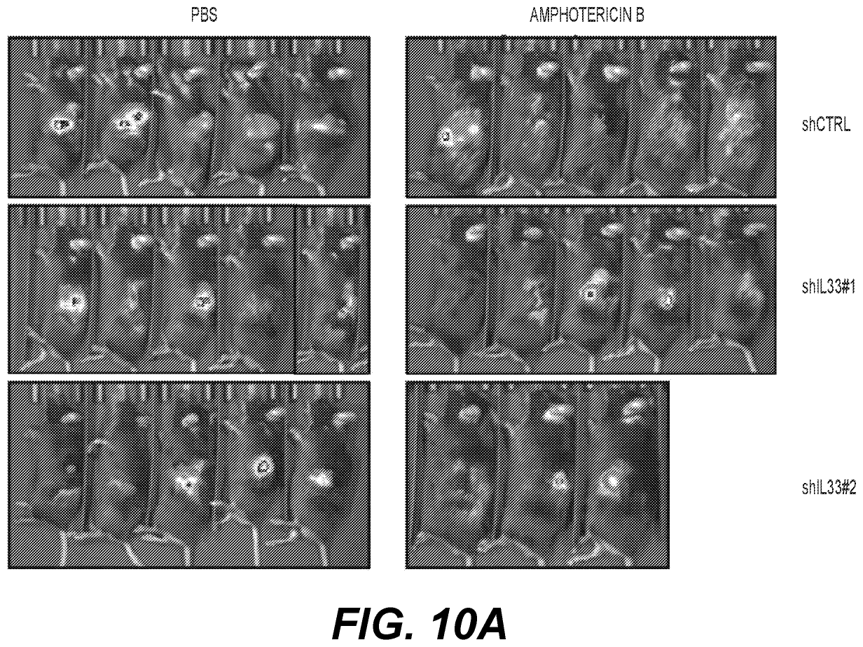

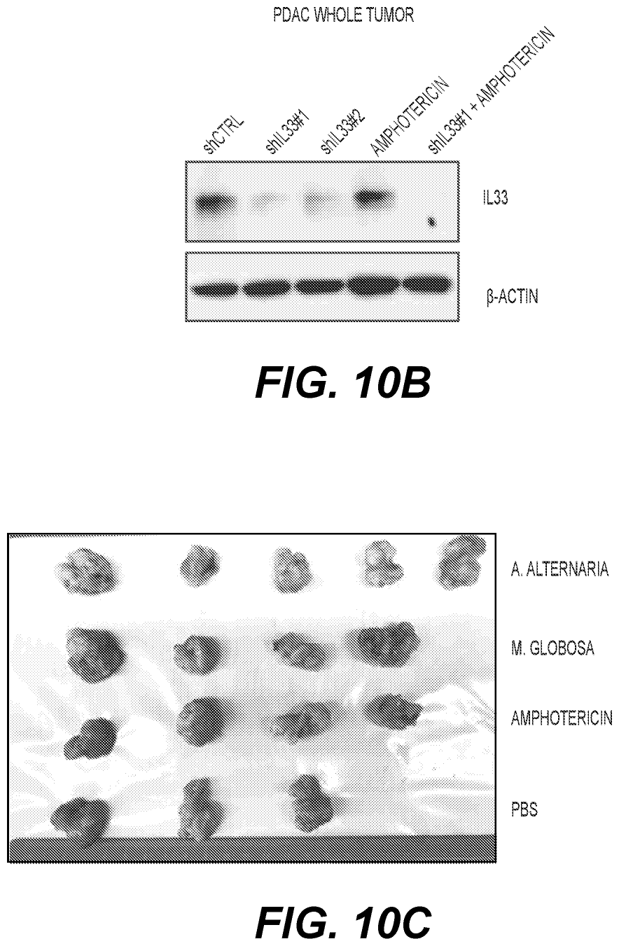

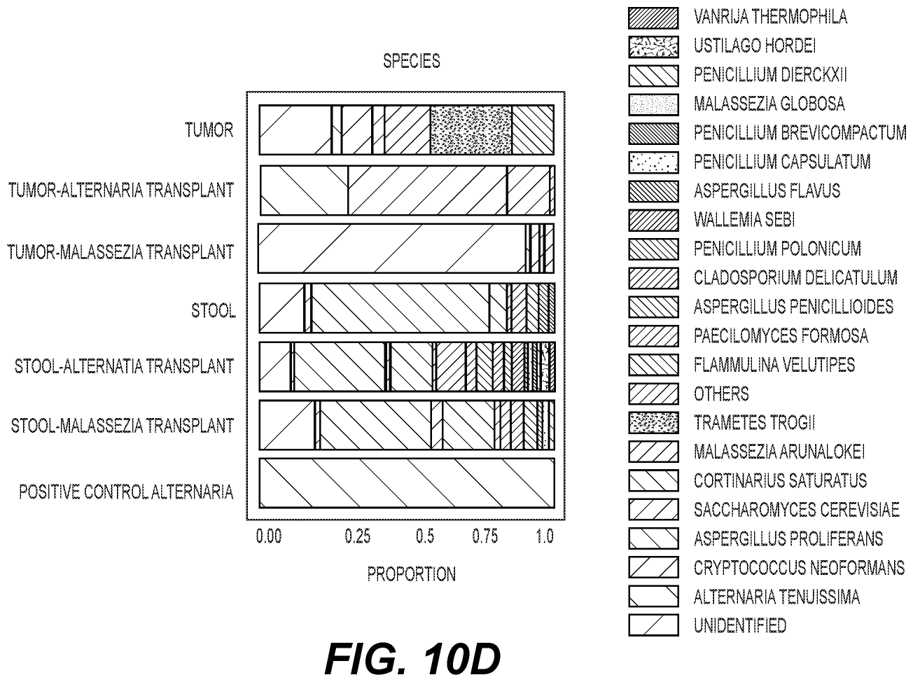

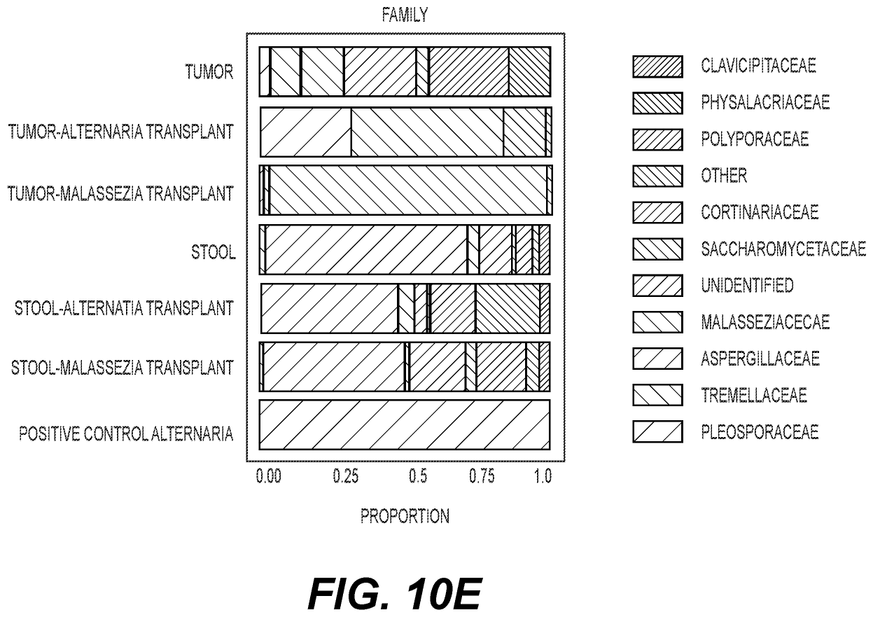

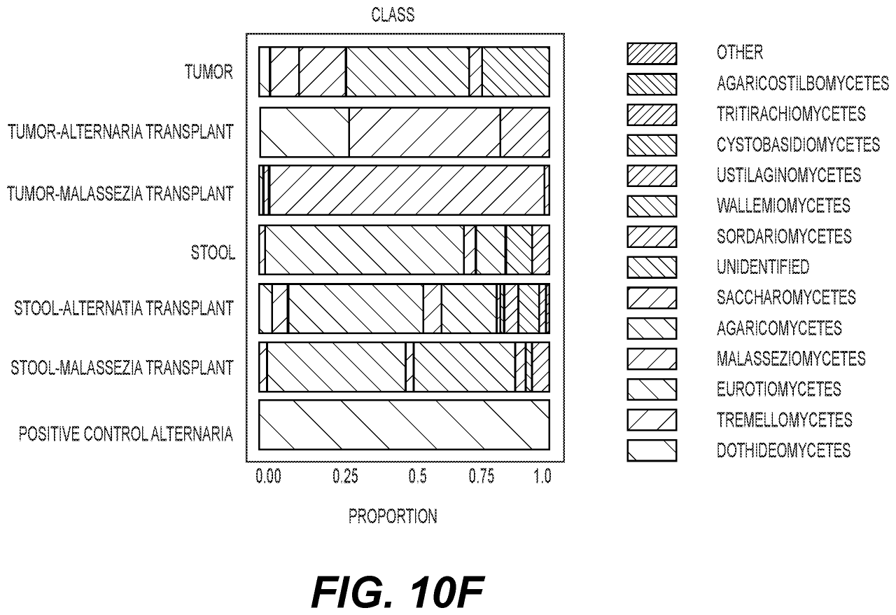

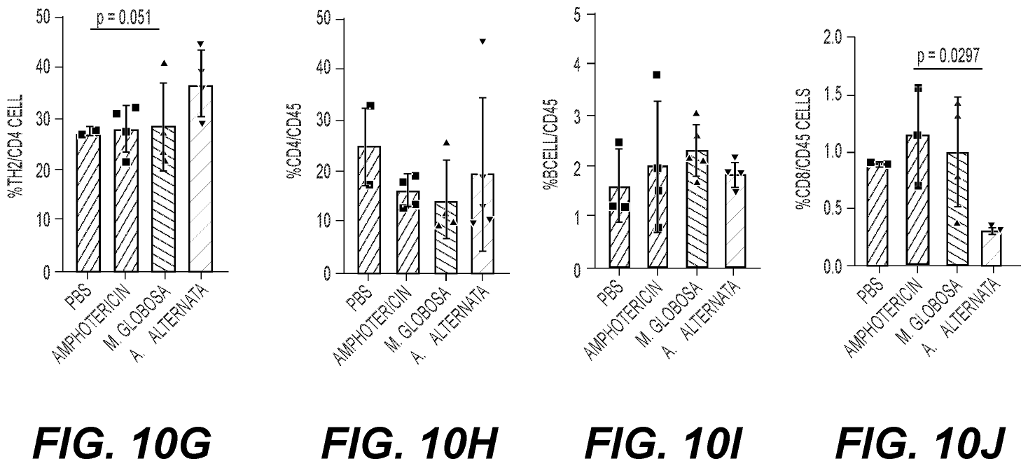

The role of fungi in promoting IL33 release by PDAC cells prompted the assessment of the impact of depleting fungi on IL33 release and the type 2 immune response in the PDAC TME. To that end, gastrointestinal fungi in tumor-bearing mice were depleted by oral amphotericin B (anti-fungal) treatment (FIG. 9A). Amphotericin B treatment or IL33 depleton resulted in decreased tumor burden and increased survival (FIG. 9B-D, FIG. 10A) as well as reduced tumor infiltrating ILC2 (FIG. 9E) and T_{H}2 cells (FIG. 9F).

To further test the role of fungi in IL33 secretion and recruitment of ILC2 and T_{H}2 cells in the PDAC TME, we evaluated the effect of Alternaria and Malassezia administered by gavage to PDAC tumor-bearing mice (FIG. 9G). First, fungi resident in the mice were depleted by a course of amphotericin B (i.e., 5 doses of amphotericin B, 200 μg/day by oral gavage), followed by a maintenance dose of 0.5 μg/ml in the drinking water for 20 days. Following depletion of fungi, we administered either Malassezia globosa or Alternaria alternata (10⁸ fungal spores per mouse) by oral gavage into the tumor-bearing mice. At 28 days post-fungal treatment, the tumors were harvested for analyses. IHC and IF showed that fungal-depleted PDAC tumors exhibited IL33 signal restricted predominantly to the nucleus of cancer cells; whereas fungi-administered PDAC tumors showed extracellular expression of IL33 (FIG. 9H). Intratumoral fungal depletion and repopulation were confirmed by fungal FISH analysis (FIG. 9H). Further, 18S rRNA sequencing confirmed increased presence of Alternaria and Malassezia sp. in the tumor and stool of the mouse receiving fungal transplantation (FIG. 9I, FIG. 10D-F). Notably, amphotericin B treatment significantly decreased tumor burden; whereas the administration of either Malassezia globosa or Alternaria alternata promoted tumor growth (FIG. 9J). Similarly, Malassezia globosa or Alternaria alternata administration augmented the infiltration of ILC2 (FIG. 9K) and T_{H}2 (FIG. 10G) cell populations within the TME. Moreover, Alternaria alternata administration decreased CD8⁺ T cells, and no significant changes in total CD4⁺ and B cells are observed (FIG. 10H-J).

### (6) IL33-Mediated ILC2 Recruitment is Necessary for Tumor Progression

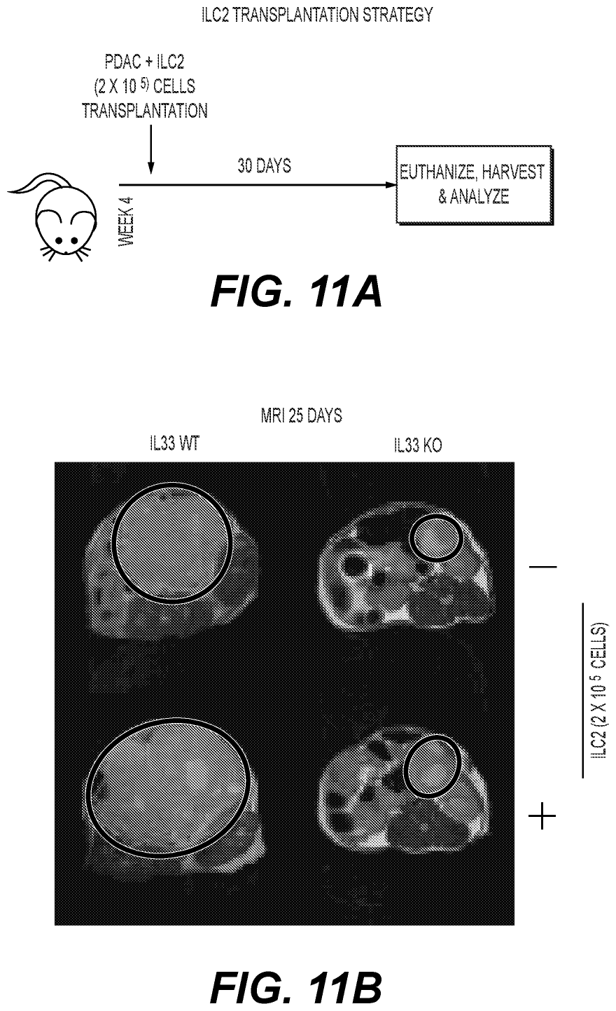

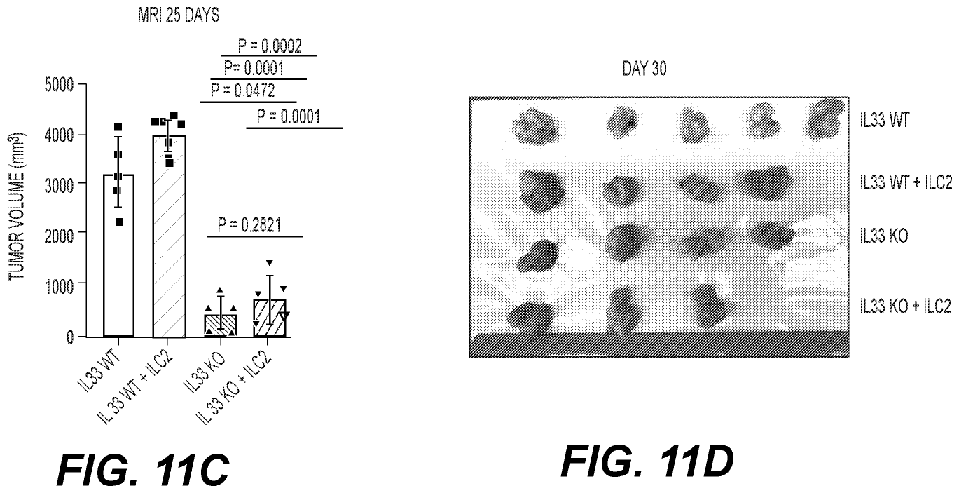

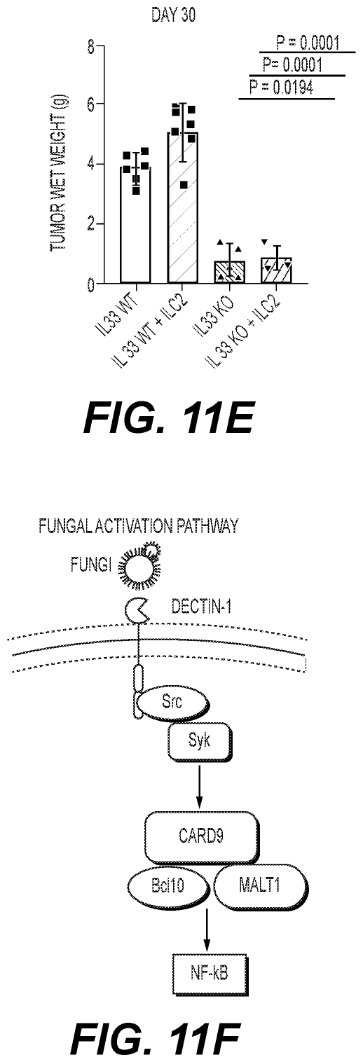

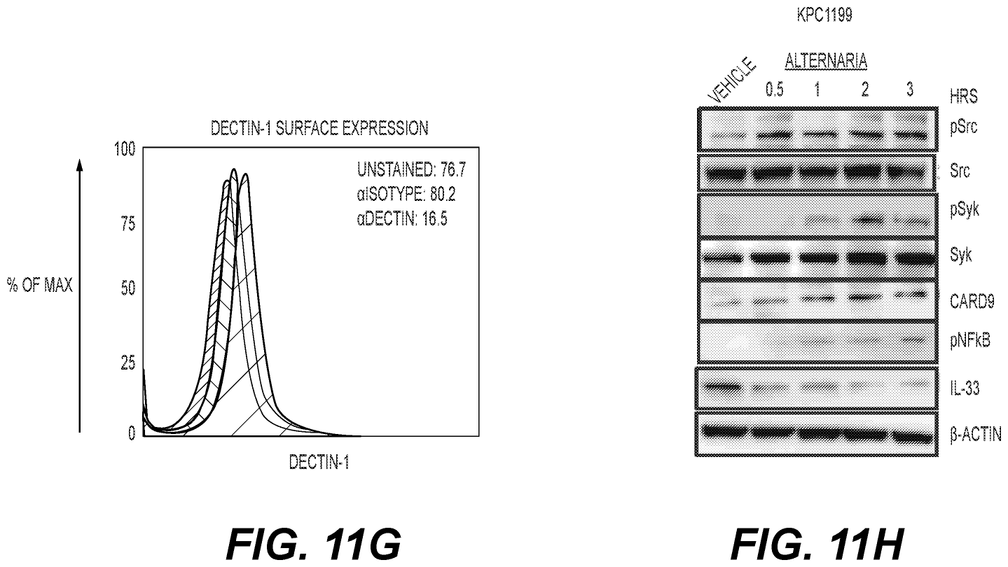

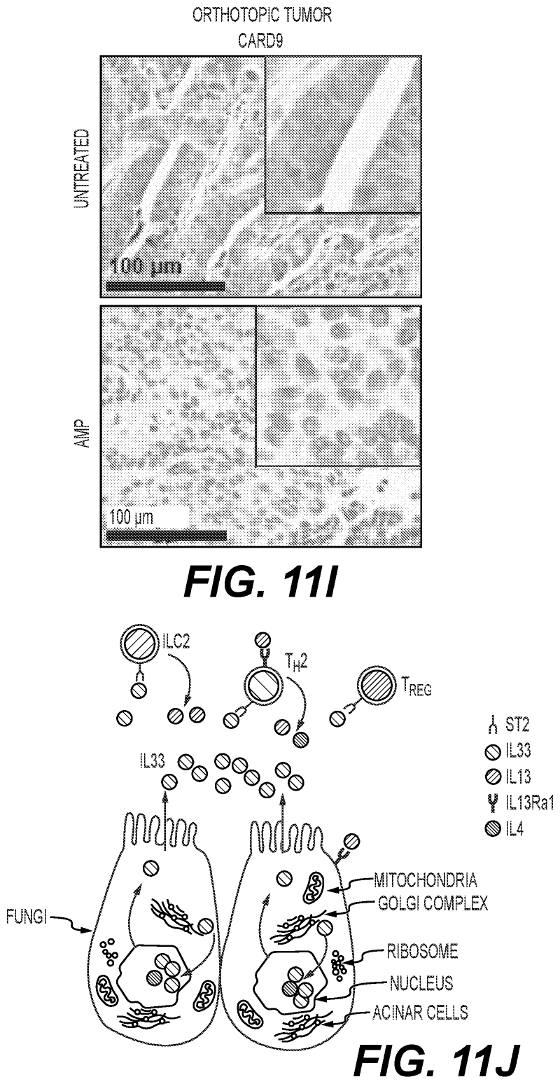

To establish the direct link between IL33-mediated ILC2 recruitment and tumor progression, we conducted an ILC2 transplantation experiment in tumor-bearing mice. First, to ensure the proper transfer of the retro-orbitally injected ILC2 cells to the PDAC TME, we established a protocol in which we labelled ILC2 cells with a Vybrant-DiD dye. The DiD-labeled ILC2 cells were then injected retro-orbitally and 7 days post-injection, the PDAC tumors were collected and DiD-labelled ILC2 cells were measured by flow cytometry (FIG. 11A). The assay confirmed the presence of DiD-labelled ILC2 cells in the PDAC tumor, as shown by the mean fluorescence intensity (MFI) levels of the ILC2 cells (FIG. 11B-C). Thereafter, we transplanted ILC2 (1×10⁵ cells) to either IL33-WT or IL33-CRISPR/Cas9 knockout PDAC tumor-bearing mice. ILC2 tranplantation in IL33-WT mice lead to a significant increase in tumor growth, whereas, tumors with CRISPR/Cas9 knockout of IL33 showed minimal change in tumor growth (FIG. 11D-F). Based on these findings, we conclude that intratumoral fungi or fungal products prime IL33 secretion by PDAC cells that promotes type 2 immune responses and tumor progression (FIG. 11G).

### b) Discussion

PDAC tumors are infiltrated by pro-tumorigenic immune cells that include T_{H}2 and ILC2 cells, which via their cytokine networks, foster a pro-tumorigenic program that leads to PDAC progression. However, it was unknown what factors mediate the recruitment and activation of T_{H}2 and ILC2 cells. In this study, we establish that Kras* regulates the expression of a chemoattracting cytokine, IL33, that recruits T_{H}2 and ILC2 cells. The T_{H}2 and ILC2 cells via their pro-tumorigenic cytokine production accelerates PDAC tumor progression. Specifically, we established IL33 as a bonafide downstream target of Kras* and that the expression of IL33 is significantly upregulated in PDAC patients. We also unraveled a novel function of an intratumoral mycobiome in facilitating the type 2 immune response in the PDAC TME by stimulating the extracellular release of IL33. The knowledge about the mechanism of extracellular release of IL33 is critical, as the inhibition of its release is amenable for targeting of the IL33-ILC2-T_{H}2 axis in PDAC-based therapy. Specifically, in a proof-of-concept study, we establish that genetic deficiency of IL33 or an anti-fungal treatment decreases tumor burden.

The mutant Kras* is the primary oncogenic driver for PDAC initiation and maintenance and, therefore, efforts have been devoted to targeting Kras*. Type 2 cytokines play a trophic role in PDAC progression and that Kras* facilitates this process by upregulating type 2 cytokine receptors—IL4Rα, IL2Rγ and IL13Rα1. Here, in addition to the Kras* mediated IL33 regulation, we demonstrate that the intratumoral mycobiome-mediated pathways stimulate PDAC cells to secrete IL33. Although the direct molecular link between fungal components and IL33 release remains to be determined, the study has revealed an important connection between the intratumoral mycobiome and the spatiotemporal release of IL33 in the PDAC TME. In addition to fungi, biochemical factors, such as ROS and oxidative stress, have been shown to promote the extracellular release of IL33. These aforementioned factors can act in parallel to the mycobiome as a stimulator of IL33 release. Notably, we and others have found that fungal components can be detected in the early stages of PDAC tumorigenesis, such as in PanIN when a large scale oxidative stress is yet to be detected in the TME.

Recent studies using metagenomic sequencing have demonstrated the presence of intratumoral fungi in PDAC. A possible route for microbial migration from the duodenum to the pancreas is the retrograde transfer via the opening of the sphincter of Oddi, which controls the flow of digestive juices (bile and pancreatic) from the pancreas and gall bladder. We have validated this finding in a mouse model where we found that PDAC tumors are infiltrated mostly by fungal genera such as Alternaria and Malassezia. The question is how soon during PDAC tumorigenesis does this retrograde transfer of fungi occur. The chain of events are crucial to understanding the mechanism of the release of IL33 into the extracellular space, the recruitment and activation of ILC2 and T_{H}2 cells and the release of pro-tumorigenic cytokines.

IL33 plays a predominant role in PDAC progression, as its deletion leads to significant tumor regression and increased survival. However, the role of IL33 is context-dependent and sometimes with opposite effects in various cancer types. Moreover, IL33 is involved in a myriad of functions depending on the spatial context of the protein. For example, the immune function of IL33 described here is distinct from its tumor cell-intrinsic function that has been described recently, in which IL33 mediates a pancreas tissue injury program in Kras mutant mice. Importantly, IL33 has been shown to cooperate with mutant Kras to initiate pancreatic neoplasia by a chromatin switch.

Finally, the temporal activation and recruitment of type 2 immune cells, especially ILC2s, are crucial for PDAC tumorigenesis. The ILC2s are mostly tissue-resident innate lymphocytes; however, recent studies show that ILC2s can be recruited from the periphery. Given the divergent function of ILC2s in different tumor types and also the context-dependent role of ILC2 in tumor progression, further study is necessary to tease apart the role of ILC2 cells in PDAC and other cancers. For example, mice lacking ST2 (IL33 receptor) have slower tumor progression by increasing T_{H}1 and NK cell activity, hinting towards a pro-tumorigenic role of an IL33-ILC2 axis. By contrast, a recent study found that the expression of PD-1 by ILC2 cells is an exploitable vulnerability and can be leveraged in PDAC patients by combining a recombinant IL33 regimen with an anti-PD-1 therapy. Therefore, it is likely that ILC2 plays a variable role in the early and late stages of the tumorigenesis. Seen in this light, the effects of fungi in priming the IL33-ILC2 axis can have different consequences based on the tumor type, location, and extent of tumor burden, and the involvement of fungi adds another layer of complexity to the IL33-ILC2 axis.

### c) Methods

### (1) Ethics Statement and Animal Modeling

All animal procedures were approved by Institutional Animal Care and Use Committee (IACUC) at Roswell Park Comprehensive Cancer Center. All animals were maintained in pathogen-free conditions and cared for in accordance with the International Association for Assessment and Accreditation of Laboratory Animal Care policies and certification. All surgeries were performed with isoflurane anesthesia. Analgesic was administered after surgery along with temperature-controlled post-surgical monitoring to minimize suffering. TetO_Lox-Stop-Lox-KrasG12D (tetO_KrasG12D), ROSA26-LSL-rtTA-IRES-GFP (ROSA_rtTA), Ptfla-Cre, LSL-Trp53, KrasG12D, Trp53R172H and pdxl-Cre strains were described previously. Mice were backcrossed to the C57BU/6 background for more than 8 generations to achieve a pure B6 mouse, and the purity and zygosity of iKPC mouse was validated by Charles River. Mice with spontaneous pancreatic tumors were euthanized at designated time points for tumor collection. Owing to the internal location of these tumors, we used signs of lethargy, reduced mobility, and morbidity, rather than maximal tumor size, as a protocol-enforced end point.

### (2) Mice and Tumor Models

For all experiments, C57BL/6J (Stock 000664) mice, aged 4-6 weeks were obtained from Jackson Laboratory unless otherwise mentioned. For orthotopic pancreas transplantation, mice were anaesthetized using isoflurane. A 2×2-mm portion of the left abdomen was shaved to facilitate transplantation. An incision was made in the left abdomen and the pancreas was gently exposed along with the spleen. Luciferase-expressing cells were slowly injected into the tail of the pancreas using a Hamilton syringe. Twenty microliters of cells (5×10⁵) mixed with 20 μl Matrigel were injected. For the orthotopic model, animals were imaged (IVIS Spectrum, PerkinElmer) 2 days after surgery to assess successful implantation of the tumors. Only orthotopic tumors of similar luciferase intensity were used further for the study. These criteria were pre-established. Furthermore, MRI and live imaging were used to monitor the progress of the tumor at different time points. For iKPC cell line Dox food treatment was started after transplantation. Owing to the internal location of the tumors, we used signs of lethargy, reduced mobility, and morbidity, rather than maximal tumor size, as a protocol-enforced end point.

(3) In vivo Imaging

Live in vivo imaging was performed at the Animal Imaging Facility at Roswell Park Comprehensive Cancer Center. Magnetic resonance imaging (MRI) was performed using a 4.7 Tesla, preclinical scanner using the ParaVision 3.0.2 platform and a 35 mm I.D. radiofrequency coil (Bruker Biospin, Billerica, MA). Mice were anesthetized with isoflurane. Temperature and respiration were regulated using an MR-compatible small animal monitoring system (Model 1250, SAII, Stony Brook, NY.) Following scout scans, tumor burden was determined using multislice, fast spin echo scans in the axial and coronal planes. Both sets of images used a TE/TR=40/2500 ms, an echo train length=8, slice thickness=1 mm, acquisition matrix=256×192 and 4 averages/NEX. Coronal images were acquired with a field of view (FOV)=48×32 mm, while axial images were acquired with a FOV=32×32 mm. A subset of axial scans required a larger number of slices to capture tumor growth, which increased the repetition time to 3600 ms. Tumor volumes were calculated by manual segmentation and voxel summation using commercially available, medical image processing software (Analyze 10.0, AnalyzeDirect, Overland Park, KS).

For bioluminescent imaging, animals were anesthetized with isoflurane, injected intraperitoneally with 3 mg of D-Luciferin (Perkin Elmer) and imaged using IVIS Spectrum Imaging System (Perkin Elmer). The Living Image 4.7 software (Perkin Elmer) was used for analysis of the images post acquisition.

(4) Transcriptomic Profiling by RNA-Seq and qRT-PCR

RNA was isolated using Trizol extraction followed by purification with the Qiagen RNAeasy kit as described previously. RNA-seq was performed by the Sequencing and Microarray Facility (SMF) core at Roswell Park Comprehensive Cancer Center. Libraries were generated using Illumina's TruSeq kit and were sequenced using the Illumina HiSeq2000 Sequencer. Raw read RNA-seq data were mapped to hg19 reference genome using Bowtie. The mapped reads were then assembled by Cufflinks to generate a transcriptome assembly for each sample. After the assembly phase, Cufflinks quantified expression level of the transcriptome in each gene for each sample (i.e., FPKM, fragments per kilobase of transcript per million fragments mapped). For qRT-PCR, RNA samples were reverse transcribed into cDNA using the High-Capacity cDNA Reverse Transcript kit (Life Technologies). cDNA samples were subjected to qRT-PCR quantification in duplicates using Power SYBR Green PCR Master Mix (Life Technologies) according to the product guides on an Agilent Mx3005P and Applied Biosystems AB7500 Fast Real Time machine. The primer sequences used for qRT-PCR are the following: IL33 (Fwd 5′ TGAGACTCCGTTCTGGCCTC 3′)(SEQ ID NO: 1), Rev 5′ CTCTTCATGCTTGGTACCCGAT 3′)(SEQ ID NO: 2), ACTB (Fwd 5′ GGCTGTATTCCCCTCCATCG 3′)(SEQ ID NO: 3), Rev 5′ CCAGTTGGTAACAATGCCATGT 3′))(SEQ ID NO: 4), Tph1 (Fwd 5′ CACGAGTGCAAGCCAAGGTIT 3′)(SEQ ID NO: 5), Rev 5′ AGTITCCAGCCCCGACATCAG 3′))(SEQ ID NO: 6), IL13 (Fwd 5′ TGAGGAGCTGAGCAACATCACACA 3′)(SEQ ID NO: 7), Rev 5′ TGCGGTITACAGAGGCCATGCAATA 3′))(SEQ ID NO: 8), IL5 (Fwd 5′ TCACCGAGCTCTGTIGACAA 3′)(SEQ ID NO: 9), Rev 5′ CCACACTTCTCTITITTGGCG 3′))(SEQ ID NO: 10), and Amphiregulin (AREG) (Fwd 5′ GGTCTTAGGCTCAGGACATTA 3′)(SEQ ID NO: 11), Rev 5′ CGCTTATGGAAACCTCTC 3′))(SEQ ID NO: 12).

### (5) Single Cell RNA Sequencing and Analyses

Single-cell transcriptomic amplification and library prep was performed using the SureCell WTA 3′ Library Prep Kit for the ddSEQ System and as previously described. Quality analysis and quantification of cDNA libraries was performed on an Agilent 2200 Tapestation system (Tapestation) using a High Sensitivity D5000 screentape (Agilent). Libraries were sequenced using a NextSeq 500 High Output Kit (Illumina). For a detailed protocol of sample preparation and analysis, refer to Bernard et al. 2018, CCR. Digital microdissection of single barcoded cells determined to be lymphocytes from overall tumor cell populations samples was performed based on expression of cell specific lineage markers of individual cells. Location of single cells representing gene expression of interest was visualized on a dimensional reduction plot utilizing FeaturePlot. All t-SNE and heat maps were run in R v3.4.2.

### (6) Flow Cytometry

Pancreas tumor single cells were isolated using the Mouse Tumor Dissociation kit (cat #130-096-730, Miltenyi Biotec). Cells from spleen were isolated by mincing with a 5-mL syringe plunger against a 70 μm cell strainer into a 60 mm dish with Roswell Park Memorial Institute (RPMI) medium containing 10% fetal bovine serum (FBS). The cells were depleted of erythrocytes by hypotonic lysis. Peripheral blood (100 μL) was drawn using retroorbital bleeding and depleted of erythrocytes by hypotonic lysis. Next, tumor, spleen or blood cells were incubated with CD16/CD32 antibody (clone 2.4G2, BD Biosciences) to block FcγR binding for 10 minutes then with antibody mix for 30 minutes at room temperature. Fluorochrome-conjugated antibodies against CD45 (clone 30-F11), CD11b (M1/70), Gr-1 (RB6-8C5), Ly-6C (HK1.4) were purchased from eBiosciences. Antibody against T-bet (644815), Lin-ve (133311), B220 (103247), CD69 (104531), CD11b (101237), ki67 (350521), IL2 (503829), KLRG1 (138419), ICOS (313533), GR1 (108405), CD45RB (151607), EpCAM (118207), IFNg (505805), CD62L (104405), CCR3 (144509), CD90.2 (140303), CD25 (102027), Lag3 (125212), CD103 (121415), CD25 (102027), CD11c (117307), CD44 (103023), Tim3 (134003), IL4Ra (144803), CTLA4 (106305), CCR4 (131203), CD117 (105807), F4/80 (123109), CD86 (105016), CD127 (135015), CD40 (124621), ST2 (146609), Ly-6c (128015), CD3 (100235), CD80 (104713), Sca1(108111), CD45 (103115), and Gata3 (16E10A23), Ly-6G (1A8), CD4 (GK1.5), CD3 (145-2C11), CD8 (53-6.7) were purchased from BioLegend. To assess cell viability, cells were incubated with Zombie UV (423107, Biolegend), dye violet (biolegend) prior to FACS analysis. All samples were acquired with the BD LSR analyzer (Becton Dickinson) and analyzed with FlowJo software (Tree Star).

### (7) Cell Culture and Establishment of Primary PDAC Lines

Primary mouse cell lines were established in the laboratory (PJ-B6-4291, PJ-B6-4298) or gift from Dr. Ronald DePinho (AK-B6, AK14838, AK192, HY19636, HY15559) as described previously and were routinely cultured in RPMI 1640 (Invitrogen) 10% FBS (Invitrogen), 100 U/ml penicillin and 100 U/ml streptomycin. For inducible Kras derived cell lines, 1 μg/ml of doxycycline was directly added to the media. The cell lines were mycoplasma free, based on tests done monthly in the laboratory using Lonza's MycoAlert Mycoplasma Detection Kit assays with confirmatory tests by PCR-based assays.

(8) shRNA and CRISPR-Cas9 Knockdown

shRNA knockdown was performed as described previously. We screened 3-5 hairpins targeting the gene of interest and found three independent sequences that reduced mRNA levels by >60%. The shRNA sequences were as follows: IL33 5′ CCGGGCATCCAAGGAACTTCACTITCTCGAGAAAGTGAAGTTCCTTGGATGCITI TG 3′ (TRCN0000173352) (SEQ ID NO: 13) and 5′ CCGGCCATAAGAAAGGAGACTAGTTCTCGAGAACTAGTCTCCTITCTTATGGTITIT TG3′ (TRCN0000176387) (SEQ ID NO: 14); 3′. A non-targeting shRNA (shCtrl) was used as a control. The shRNA-expressing pLKO.1 vector was introduced into cancer cell lines by lentiviral infection. Recombinant lentiviral particles were produced by transient transfection of 293T cells following a standard protocol. Briefly, 10 μg of the shRNA plasmid, 5 μg of psPAX2 and 2.5 μg of pMD2.G were transfected using Lipofectamine 3000 (Invitrogen) into 293T cells plated in a 100-mm dish. Viral supernatant was collected 72 h after transfection, centrifuged to remove any 293T cells and filtered (0.45 μm). For transduction, viral solutions were added to cell culture medium containing 4 μg/ml polybrene; 48 h after infection, cells were selected using 2 μg/ml puromycin and tested for gene depletion by qRT-PCR or immunoblotting. For CRISPR knockdown of IL33, sgRNAs were purchased from Synthego (Sanger CRISPR clones). The sgRNAs along with Cas9 protein (sigma) was transfected into PDAC cells and single cell clones were facs sorted. The sgIL33 sequence used for IL33 DNA sequence targeting. 5′ AUAGUAGCGUAGCGUAGUAGCACC 3′ (SEQ ID NO: 15) and 5′ AUCUCUUCCUAGAAUCCCG 3′(SEQ ID NO: 16). The clones were validated by western blot for deletion of IL33.

### (9) Immunoblotting and Antibodies

Medium was removed and the cells were washed twice in ice-cold phosphate-buffered saline (PBS), scraped and collected as pellets after centrifugation at 1700 g for 5 min. The pelleted cells were incubated in radio immune precipitation assay (RIPA) buffer with proteinase and phosphatase inhibitors for 15 min. Lysates were then collected and centrifuged at 12000 rpm for 15 min at 4° C. Protein concentrations were measured using the DC Protein Assay Kit (Biorad, Cat. No. 5000111). SDS-PAGE and immunoblotting were performed as described previously in pre-cast bis-Tris 4-20% gradient gels (Invitrogen). The following antibodies were used: IL33 (R & D system AF3626); pAKT-S473 (CST 9271); pERK-p44/42 (CST 4370) and P-Actin (Sigma-Aldrich, A2228).

### (10) Immunohistochemistry and Immunofluorescence

Harvested tissues were immediately fixed in 10% formalin overnight and embedded in paraffin. IHC was performed as described previously. Briefly, endogenous peroxidases were inactivated by 3% hydrogen peroxide. Non-specific signal was blocked using 5% BSA for 30 mins in 0.1% Tween 20. Tumor samples were stained with the following primary antibodies: CD45 (Abcam ab10558), mouse IL33 antibody (R&D system AF3626); human IL33 antibody (R&D system AF3625); and); anti human IL33 (R&D system AF3626) and Pan-Keratin (C11) (CST 4545S). After overnight incubation, the slides were washed and incubated with secondary antibody (HRP-polymers, Biocare Medical) for 30 min at room temperature. The slides were washed three times and stained with DAB substrate (ThermoFisher Scientific). The slides were then counterstained with haematoxylin and mounted with mounting medium. Immunofluorescence slides were imaged with an Leica confocal Microscope and quantified with ImageJ.

### (11) Human PDAC Primary Tumor Samples

Human PDAC samples were obtained from Roswell Park Comprehensive Cancer Center's tissue Biobank. The samples were stained using the standard IHC protocol. The antibodies used were IL33 (R&D system AF3626) and secondary antibody (HRP-polymers, Biocare Medical). The stained samples were imaged using Aperio ScanScope XT scanner and Aperio ImageScope software was used for image visualization. Data was analyzed using Pannoramic viewer software (3DHISTECH Ltd). Staining intensity of tissue sections was scored in a ‘blinded’ manner by a pathologists. Human studies were approved by Roswell Park Comprehensive Cancer Center's Institutional Review Board, and prior informed consent was obtained from all subjects under IRB protocol STUDY00001407/BDR 135920.

### (12) Antifungal Treatment and Fungal Challenge Experiments

For gastrointestinal fungal depletion, C57BL/6 mice were treated with 200 μg amphotericin B per day by oral gavage for five consecutive days, followed by 0.5 μg/ml amphotericin B treatment in drinking water for 21 days. Control groups were gavaged with 200 μl PBS for 5 consecutive days. After completion of antifungal treatment, species specific fungal repopulation was done with Alternaria alternata (ATCC 36376) and Malassezia globosa (MYA-4889). Fungi were administered (1×10⁸ CFU/ml) by oral gavage. Seven days after fungal administration, AK-B6 PDAC cells were orthotopically transplanted.

### (13) Fungal Fluorescence In-Situ Hybridization (FISH)

FISH was done on a 4 μm thick paraffin embedded pancreatic tissue sections. Sections were pretreated using a commercially available kit (Cytocell, Inc) according to manufacturer's instructions. For Hybridization D223 28S rRNA gene probe labeled with the 6-FAM fluorophore (extinction wavelength, 488 nm; emission wavelength, 530 nm) was used to detect the fungal colonization within mouse pancreatic tissues. Hybridization and post hybridization washes were conducted according to standard procedures. Slides were visualized on an Olympus BX61 microscope.

(14) 18S rRNA Sequencing and Mycobiome Analysis

The sequencing libraries were prepared using a two-step PCR method using the primer set ITSIF (5′-CTTGGTCATITAGAGGAAGTAA-3′)(SEQ ID NO: 17) and ITS2 (5′-GCTGCGTTCTTCATCGATGC-3′)(SEQ ID NO: 18). The first PCR (25-cycle) uses 25 ng of DNA to amplify the target region, where the PCR primers have overhang adapter sequence necessary for the second PCR step. After purification, the amplicon from the first step is amplified with 8 cycles of PCR using the Nextera Index Kit (Illumina Inc.), which uses primers that target the overhang adaptor sequence added during the first round of PCR. The second round PCR adds one of 384 different combinations of indexed tags to each sample which allows pooling of libraries and multiplex sequencing. Prior to pooling, each individual sample's amplified DNA is visualized on a Tapestation 4200 D1000 tape (Agilent Technologies) for expected amplicon size, purity and concentration. Validated libraries are pooled equal molar in a final concentration of 4 nM in Tris-HCl 10 mM, pH 8.5, before 2×300 cycle sequencing on a MiSeq (Illumina, Inc.).

Paired-end fastq reads were demultiplexed, processed and analyzed using QIIME (v1.9.1). Then, operational taxonomic units (OTUs) were assigned using QIIME's uclust-based open-reference OTU-picking pipeline. OTUs with less than 0.001% assigned sequences were removed from each sample to avoid biased and inflated diversity estimates. ITS samples were processed following the QIIME pipeline steps using UNITE's Fungi taxon (v8.4) reference-annotation adapted for QIIME. Chimeras were removed before taxonomy assignments with vsearch (v2.15.0) using the UCHIME reference dataset (v7.2) available at the UNITE website. Positive and negative control samples were checked for QC purposes.

Taxonomy assignments from all samples were then compiled in a raw-counts matrix. Raw counts were formatted, processed, and analyzed using phyloseq package (v1.28.0) in R (v4.0.0). 16S data is summarized to OTUs at the genus level. Observed, Chaol, Shannon and Simpson's Reciprocal diversity indices were estimated for alpha-diversity scores; mean estimates were obtained performing 100 bootstrapped rarefactions. Same analyses were performed for ITS, but at the species and genus levels. For Beta-diversity, Bray-Curtis dissimilarity score paired with classical multidimensional scaling was estimated and plotted using the vegan package (v2.5.6).

### (15) Enzyme Linked Immunosorbent Assay (ELISA)

ELISA was done using ascitic fluid of PDAC tumor bearing mice and PDAC mouse cell culture conditioned media. Cell culture media was concentrated using Amicon Ultra centrifugal filter units (Millipore, Z717185). IL33 ELISA was performed using LEGEND MAX™ Mouse IL-33 ELISA Kit (Biolegend) using manufacturers standard protocol. IL5 ELISA was performed using Mouse IL-5 Quantikine ELISA Kit (R&D system) using manufacturers standard protocol.

### (16) ILC2 Enrichment and Adoptive Transfer of ILC2

Orthotopically transplanted PDAC tumors were chopped and single cell suspension was prepared using mouse tumor dissociation Kit (Milteny biotech). Ficoll column was used to separate the immune cells. ILC2 was purified using ILC2 enrichment kit (Stem cells Technology) using standard protocol. Orthotopically transplanted PDAC tumor bearing mice were used for adoptive transfer experiments. After 10 days of orthotopic pancreatic injections of PDAC cells, 1×10⁵ILC2 were adoptively transferred via retro-orbital injection and were monitored for tumor progression. For localization of adoptively transferred ILC2 in the orthotopically transplanted mouse tumor, ILC2 was labelled with a lipid binding DiD′ solid (1,1′-Dioctadecyl-3,3,3′,3′-Tetramethylindodicarbocyanine, 4-Chlorobenzenesulfonate Salt (Invitrogen, D7757). After 7 days, PDAC tumor is harvested and DiD-labelled ILC2 was measured by flow cytometry using APC channel.

### (17) Statistical Analysis

GraphPad Prism software was used to conduct the statistical analysis of all data except for qRT-PCR data, where Microsoft excel was used. Data are presented as mean t SEM. All quantitative results were assessed by unpaired Student's t-test after confirming that the data met appropriate assumptions (normality and independent sampling). The Student t-test assumed two-tailed distributions to calculate statistical significance between groups. Unless otherwise indicated, for all in vitro experiments, three technical replicates were analyzed. Sample size estimation was done taking into consideration previous experience with animal strains, assay sensitivity and tissue collection methodology used. Animal survival impact was determined by the Kaplan-Meier analysis. P<0.05 was considered statistically significant; the P values are indicated in the figures.

### E. SEQUENCES

IL33 Forward Primer
SEQ ID NO: 1
TGAGACTCCGTTCTGGCCTC
IL33 Reverse Primer
SEQ ID NO: 2
CTCTTCATGCTTGGTACCCGAT
ACTB Forward Primer
SEQ ID NO: 3
GGCTGTATTCCCCTCCATCG
ACTB Reverse Primer
SEQ ID NO: 4
CCAGTTGGTAACAATGCCATGT
Tph1 Forward Primer
SEQ ID NO: 5
CACGAGTGCAAGCCAAGGTTT
Tph1 Reverse Primer
SEQ ID NO: 6
AGTTTCCAGCCCCGACATCAG
IL13 Forward Primer
SEQ ID NO: 7
TGAGGAGCTGAGCAACATCACACA
IL13 Reverse Primer
SEQ ID NO: 8
TGCGGTTACAGAGGCCATGCAATA
IL5 Forward Primer
SEQ ID NO: 9
TCACCGAGCTCTGTTGACAA
IL5 Reverse Primer
SEQ ID NO: 10
CCACACTTCTCTTTTTGGCG
Amphiregulin (AREG) Forward Primer
SEQ ID NO: 11
GGTCTTAGGCTCAGGACATTA
Amphiregulin (AREG) Reverse Primer
SEQ ID NO: 12
CGCTTATGGAAACCTCTC
IL33 shRNA sequence
SEQ ID NO: 13
CCGGGCATCCAAGGAACTTCACTTTCTCGAGAAAGTGAAGTTCCTTGGAT
GCTTTTTTG
IL33 shRNA sequence
SEQ ID NO: 14
CCGGCCATAAGAAAGGAGACTAGTTCTCGAGAACTAGTCTCCTTTCTTAT
GGTTTTTTG
sgIL33 sequence used for IL33 DNA
sequence targeting
SEQ ID NO: 15
AUAGUAGCGUAGCGUAGUAGCACC
sgIL33 sequence used for IL33 DNA
sequence targeting
SEQ ID NO: 16
AUCUCUUCCUAGAAUCCCG

### F. REFERENCES

Abarenkov, K., et al., The UNITE database for molecular identification of fungi—recent updates and future perspectives. New Phytol, 2010. 186(2): p. 281-5.
Aguirre, A. J., et al., Activated Kras and Ink4a/Arf deficiency cooperate to produce metastatic pancreatic ductal adenocarcinoma. Genes Dev, 2003. 17(24): p. 3112-26.
Alamri, A., J. Y. Nam, and J. K. Blancato, Fluorescence In Situ Hybridization of Cells, Chromosomes, and Formalin-Fixed Paraffin-Embedded Tissues. Methods Mol Biol, 2017. 1606: p. 265-279.
Ali, S., et al., IL-1 receptor accessory protein is essential for IL-33-induced activation of T lymphocytes and mast cells. Proc Natd Acad Sci USA, 2007. 104(47): p. 18660-5.
Alonso-Curbelo, D., et al., A gene-environment-induced epigenetic program initiates tumorigenesis. Nature, 2021. 590(7847): p. 642-648.
Aykut, B., et al., The fungal mycobiome promotes pancreatic oncogenesis via activation of MBL. Nature, 2019. 574(7777): p. 264-267.
Bernard, V., et al., Single-Cell Transcriptomics of Pancreatic Cancer Precursors Demonstrates Epithelial and Microenvironmental Heterogeneity as an Early Event in Neoplastic Progression. Clin Cancer Res, 2019. 25(7): p. 2194-2205.
Bonilla, W. V., et al., The alarmin interleukin-33 drives protective antiviral CD8(+) T cell responses. Science, 2012. 335(6071): p. 984-9.
Caporaso, J. G., et al., QIIME allows analysis of high-throughput community sequencing data. Nat Methods, 2010. 7(5): p. 335-6.
Clark, C. E., et al., Dynamics of the immune reaction to pancreatic cancer from inception to invasion. Cancer Res, 2007. 67(19): p. 9518-27.
Cui, G., et al., Contribution of IL-33 to the Pathogenesis of Colorectal Cancer. Front Oncol, 2018. 8: p. 561.
De Monte, L., et al., Intratumor T helper type 2 cell infiltrate correlates with cancer-associated fibroblast thymic stromal lymphopoietin production and reduced survival in pancreatic cancer. J Exp Med, 2011.208(3): p. 469-78.
Dey, P., A. Strom, and J. A. Gustafsson, Estrogen receptor beta upregulates FOXO3a and causes induction of apoptosis through PUMA in prostate cancer. Oncogene, 2014. 33(33): p. 4213-25.
Dey, P., A. C. Kimmelman, and R. A. DePinho, Metabolic Codependencies in the Tumor Microenvironment. Cancer Discov, 2021.
Dey, P., et al., Estrogen receptors beta) and beta2 have opposing roles in regulating proliferation and bone metastasis genes in the prostate cancer cell line PC3. Mol Endocrinol, 2012. 26(12): p. 1991-2003.
Dey, P., et al., Genomic deletion of malic enzyme 2 confers collateral lethality in pancreatic cancer. Nature, 2017. 542(7639): p. 119-123.
Dey, P., et al., Oncogenic KRAS-Driven Metabolic Reprogramming in Pancreatic Cancer Cells Utilizes Cytokines from the Tumor Microenvironment. Cancer Discov, 2020. 10(4): p. 608-625.
Eberl, G., et al., Innate lymphoid cells. Innate lymphoid cells: a new paradigm in immunology. Science, 2015. 348(6237): p. aaa6566.
Ercolano, G., et al., ILC2s: New Actors in Tumor Immunity. Front Immunol, 2019. 10: p. 2801.
Feig, C., et al., The pancreas cancer microenvironment. Clin Cancer Res, 2012. 18(16): p. 4266-76.
Flamar, A. L., et al., Interleukin-33 Induces the Enzyme Tryptophan Hydroxylase 1 to Promote Inflammatory Group 2 Innate Lymphoid Cell-Mediated Immunity. Immunity, 2020.52(4): p. 606-619 e6.
Fournie, J. J. and M. Poupot, The Pro-tumorigenic IL-33 Involved in Antitumor Immunity: A Yin and Yang Cytokine. Front Immunol, 2018.9: p. 2506.
Gopalakrishnan, V., et al., Gut microbiome modulates response to anti-PD-1 immunotherapy in melanoma patients. Science, 2018. 359(6371): p. 97-103.
Jovanovic, I., et al., ST2 deletion enhances innate and acquired immunity to murine mammary carcinoma. Eur J Immunol, 2011.41(7): p. 1902-12.
Koljalg, U., et al., Towards a unified paradigm for sequence-based identification of fungi. Mol Ecol, 2013. 22(21): p. 5271-7.
Langmead, B. and S. L. Salzberg, Fast gapped-read alignment with Bowtie 2. Nat Methods, 2012. 9(4): p. 357-9.
Li, A., et al., IL-33 Signaling Alters Regulatory T Cell Diversity in Support of Tumor Development. Cell Rep, 2019. 29(10): p. 2998-3008 e8.
Liew, F. Y., J. P. Girard, and H. R. Turnquist, Interleukin-33 in health and disease. Nat Rev Immunol, 2016. 16(11): p. 676-689.
Lingel, A., et al., Structure of IL-33 and its interaction with the ST2 and IL-1RAcP receptors-insight into heterotrimeric IL-1 signaling complexes. Structure, 2009. 17(10): p. 1398-410.
Matson, V., et al., The commensal microbiome is associated with anti-PD-1 eficacy in metastatic melanoma patients. Science, 2018. 359(6371): p. 104-108.
McMurdie, P. J. and S. Holmes, phyloseq: an R package for reproducible interactive analysis and graphics of microbiome census data. PLoS One, 2013. 8(4): p. e61217.
Miller, A. M., Role of IL-33 in inflammation and disease. J Inflamm (Lond), 2011. 8(1): p. 22.
Moral, J. A., et al., ILC2s amplify PD-1 blockade by activating tissue-specific cancer immunity. Nature, 2020. 579(7797): p. 130-135.
Oksanen, J., Kindt, R., Legendre, P., O'Hara, B., Stevens, M. H. H., Oksanen, M. J. and Suggests, M. A. S. S., The vegan package. Community ecology package. 2007(10(631-637)): p. 719.
Pastille, E., et al., The IL-33/ST2 pathway shapes the regulatory T cell phenotype to promote intestinal cancer. Mucosal Immunol, 2019. 12(4): p. 990-1003.
Piro, G., et al., A circulating TH2 cytokines profile predicts survival in patients with resectable pancreatic adenocarcinoma. Oncoimmunology, 2017. 6(9): p. e1322242.
Rank, M. A., et al., IL-33-activated dendritic cells induce an atypical TH2-type response. J Allergy Clin Immunol, 2009. 123(5): p. 1047-54.
Riquelme, E., et al., Tumor Microbiome Diversity and Composition Influence Pancreatic Cancer Outcomes. Cell, 2019. 178(4): p. 795-806 e12.
Robinette, M. L., et al., Transcriptional programs define molecular characteristics of innate lymphoid cell classes and subsets. Nat Immunol, 2015. 16(3): p. 306-17.
Rognes, T., et al., VSEARCH: a versatile open source tool for metagenomics. PeerJ, 2016. 4: p. e2584.
Schmitz, J., et al., IL-33, an interleukin-I-like cytokine that signals via the IL-1 receptor-related protein ST2 and induces T helper type 2-associated cytokines. Immunity, 2005. 23(5): p. 479-90.
Snelgrove, R. J., et al., Alternaria-derived serine protease activity drives IL-33-mediated asthma exacerbations. J Allergy Clin Immunol, 2014. 134(3): p. 583-592 e6.
Steele, C. W., et al., Exploiting inflammation for therapeutic gain in pancreatic cancer. Br J Cancer, 2013. 108(5): p. 997-1003.
Taniguchi, S., et al., Tumor-initiating cells establish an IL-33-TGF-beta niche signaling loop to promote cancer progression. Science, 2020. 369(6501).
Tibbitt, C. A., et al., Single-Cell RNA Sequencing of the T Helper Cell Response to House Dust Mites Defines a Distinct Gene Expression Signature in Airway Th2 Cells. Immunity, 2019. 51(1): p. 169-184 e5.
Trapnell, C., et al., Differential gene and transcript expression analysis of RNA-seq experiments with TopHat and Cufflinks. Nat Protoc, 2012. 7(3): p. 562-78.
Uchida, M., et al., Oxidative stress serves as a key checkpoint for IL-33 release by airway epithelium. Allergy, 2017. 72(10): p. 1521-1531.
Van Gool, F., et al., Interleukin-5-producing group 2 innate lymphoid cells control eosinophilia induced by interleukin-2 therapy. Blood, 2014. 124(24): p. 3572-6.
Varricchi, G., et al., Eosinophils: The unsung heroes in cancer? Oncoimmunology, 2018. 7(2): p. e1393134.
Venmar, K. T., et al., IL4 receptor alpha mediates enhanced glucose and glutamine metabolism to support breast cancer growth. Biochim Biophys Acta, 2015. 1853(5): p. 1219-28.
Vivier, E., et al., Innate Lymphoid Cells: 10 Years On. Cell, 2018. 174(5): p. 1054-1066.
Wang, K., et al., IL-33 blockade suppresses tumor growth of human lung cancer through direct and indirect pathways in a preclinical model. Oncotarget, 2017. 8(40): p. 68571-68582.
Ying, H., et al., Genetics and biology of pancreatic ductal adenocarcinoma. Genes Dev, 2016. 30(4): p. 355-85.
Ying, H., et al., Oncogenic Kras maintains pancreatic tumors through regulation of anabolic glucose metabolism. Cell, 2012. 149(3): p. 656-70.
Zervos, E. E., et al., Prognostic significance of new onset ascites in patients with pancreatic cancer. World J Surg Oncol, 2006. 4: p. 16.

1. A method of treating a cancer comprising a KRAS^{G12D} substitution in a subject, inhibiting T_{H}2 pro-tumorigenic cytokines in a tumor microenvironment of a cancer in a subject, decreasing secretion of IL-33 in a tumor microenvironment of a cancer in a subject, or decreasing infiltration of Type 2 immune cells in a tumor microenvironment of a cancer in a subject, the method comprising administering to the subject an antifungal agent, a MEK inhibitor, an IL-33 inhibitor, or an IL-33 receptor (suppression of tumorigenicity (ST)2) inhibitor, or any combination thereof.

2. The method of claim 1, wherein the antifungal agent inhibits Alternaria alternata and/or Malassezia globosa infection in the tumor microenvironment.

3. The method of claim 2, wherein the antifungal agent is selected from the group consisting of natamycin, hamicyn, filipinmycostatin, amphotericin B, albaconazole, efinaconazole, epoxiconazole, isavuconazole, ketoconazole, clotrimazole, posaconazole, propiconazole, ravuconazole, terconazole, miconazole, flucytosine, fluconazole, itraconazole, abafungin, micafungin, caspofungin, anidulafungin, bifonazole, butoconazole, clotrimazole, econazole, fenticonazole, isoconazole, ketoconazole, luliconazole, omoconazole, oxiconazole, sertaconazole, sulconazole, tioconazole, and voriconazole.

4. The method of claim 1, wherein the antifungal agent is administered prior to the onset of pancreatitis.

5. The method of claim 1, wherein the IL-33 inhibitor comprises an antibody, small molecule, shRNA, RNAi, CRISPR/CAS9 nuclease, TALEN nuclease, or zinc finger nuclease.

6. The method of claim 5, wherein the IL-33 inhibitor comprises an antibody selected from the group consisting of AF3625 and AF6326.

7. The method of claim 5, wherein the IL-33 inhibitor comprises an shRNA selected from the group consisting of SEQ ID NO: 13 and SEQ ID NO: 14.

8. The method of claim 5, wherein the IL-33 inhibitor comprises a CRISPR/Cas9 nuclease targeting IL-33 with single guide RNA (sgRNA) selected from the group consisting of SEQ ID NO: 15 and SEQ ID NO: 16.

9. The method of claim 1, wherein the ST2 inhibitor comprises an antibody or small molecule.

10. The method of claim 1, wherein the MEK inhibitor comprises CI-1040, PD0325901, binimetinib, cobimetinib, selumetinib, or Trametinib.

11. The method of claim 1, wherein the method further comprises administering to the subject an anti-cancer agent.

12. The method of claim 1, wherein the cancer comprises pancreatic cancer, colon cancer, and lung cancer.

13. (canceled)

14. The method of claim 1, wherein the T_{H}2 pro-tumorigenic cytokines comprises IL4, IL5 and IL13.

15-36. (canceled)

37. The method of claim 1, wherein the Type 2 immune cells comprise innate lymphoid cells (ILC) 2 (ILC2) or T_{H}2 cells.

38-47. (canceled)


SEQUENCE LISTING (16 sequences)

SEQ ID NO: 1 - DNA, 20 bp, synthetic construct
  source 1..20: mol_type = other DNA
tgagactccgttctggcctc

SEQ ID NO: 2 - DNA, 22 bp, synthetic construct
  source 1..22: mol_type = other DNA
ctcttcatgcttggtacccgat

SEQ ID NO: 3 - DNA, 20 bp, synthetic construct
  source 1..20: mol_type = other DNA
ggctgtattcccctccatcg

SEQ ID NO: 4 - DNA, 22 bp, synthetic construct
  source 1..22: mol_type = other DNA
ccagttggtaacaatgccatgt

SEQ ID NO: 5 - DNA, 21 bp, synthetic construct
  source 1..21: mol_type = other DNA
cacgagtgcaagccaaggttt

SEQ ID NO: 6 - DNA, 21 bp, synthetic construct
  source 1..21: mol_type = other DNA
agtttccagccccgacatcag

SEQ ID NO: 7 - DNA, 24 bp, synthetic construct
  source 1..24: mol_type = other DNA
tgaggagctgagcaacatcacaca

SEQ ID NO: 8 - DNA, 24 bp, synthetic construct
  source 1..24: mol_type = other DNA
tgcggttacagaggccatgcaata

SEQ ID NO: 9 - DNA, 20 bp, synthetic construct
  source 1..20: mol_type = other DNA
tcaccgagctctgttgacaa

SEQ ID NO: 10 - DNA, 20 bp, synthetic construct
  source 1..20: mol_type = other DNA
ccacacttctctttttggcg

SEQ ID NO: 11 - DNA, 21 bp, synthetic construct
  source 1..21: mol_type = other DNA
ggtcttaggctcaggacatta

SEQ ID NO: 12 - DNA, 18 bp, synthetic construct
  source 1..18: mol_type = other DNA
cgcttatggaaacctctc

SEQ ID NO: 13 - DNA, 59 bp, synthetic construct
  source 1..59: mol_type = other DNA
ccgggcatccaaggaacttcactttctcgagaaagtgaagttccttggatgcttttttg

SEQ ID NO: 14 - DNA, 59 bp, synthetic construct
  source 1..59: mol_type = other DNA
ccggccataagaaaggagactagttctcgagaactagtctcctttcttatggttttttg

SEQ ID NO: 15 - RNA, 24 nt, synthetic construct
  source 1..24: mol_type = other RNA
atagtagcgtagcgtagtagcacc

SEQ ID NO: 16 - RNA, 19 nt, synthetic construct
  source 1..19: mol_type = other RNA
atctcttcctagaatcccg